(19) United States
(12) Patent Application Publication
(10) Pub. No.: US 2024/0108612 A1
(43) Pub. Date: Apr. 4, 2024

(54) PHARMACEUTICAL COMPOSITION FOR ENHANCING ANTICANCER EFFECT OF PROTAC, INCLUDING IU1-SERIES UBIQUITIN-SPECIFIC PROTEASE 14 INHIBITOR AS ACTIVE INGREDIENT

(71) Applicant: DAEGU GYEONGBUK INSTITUTE OF SCIENCE AND TECHNOLOGY, Daegu (KR)

(72) Inventors: Byung-Hoon LEE, Daegu (KR); Seonghyeon MOON, Daegu (KR); Thuy Thi Thanh TRAN, Hanoi (VN)

(73) Assignee: DAEGU GYEONGBUK INSTITUTE OF SCIENCE AND TECHNOLOGY, Daegu (KR)

(21) Appl. No.: 18/518,727

(22) Filed: Nov. 24, 2023

Related U.S. Application Data
(63) Continuation-in-part of application No. PCT/KR22/07066, filed on May 17, 2022.

(30) Foreign Application Priority Data
May 24, 2021 (KR) 10-2021-0066398

(51) Int. Cl. A61K 31/4412; A61K 31/4439; A61K 45/06; A61P 35/00

(52) U.S. Cl. CPC A61K 31/4412; A61K 31/4439; A61K 45/06; A61P 35/00

(57) ABSTRACT

Provided is a pharmaceutical composition for enhancing the anticancer effect of cancer. More specifically, it was confirmed that when a composition including an IU1-series compound known to inhibit ubiquitin-specific protease 14 (USP14), which is a deubiquitinase, is administered in combination with and proteolysis-targeting chimera (PROTAC), the cancer prevention or treatment effect of PROTAC was significantly enhanced. Furthermore, with the pharmaceutical composition of the present disclosure, cancer can be effectively treated even when a lower dose of PROTAC is used, thereby freeing from the problems of toxicity and side effects, and improving the application limitations and problems arising from the sole use of PROTAC. Accordingly, the combinatorial treatment of PROTAC and IU1-series compound can be widely used in the field of cancer treatment.

### TECHNICAL FIELD

The present disclosure relates to a pharmaceutical composition for enhancing anticancer effects, and more specifically, to a composition for enhancing the prevention and/or treatment effect of proteolysis-targeting chimera (PROTAC) on cancer, including IU1-series compounds among ubiquitin-specific protease 14 (hereinafter referred to as USP14) inhibitors, wherein USP14 is one of the deubiquitinases (DUBs).

### BACKGROUND ART

New drugs of the related art work on the principle of inhibiting the active site or ligand binding site of a disease targeted protein, and currently, most small-molecule inhibitors and therapeutic antibodies have been developed based on this method. Although these drugs are used as effective treatments for several human diseases, including cancers, there are still an overwhelming number of diseases and disease-target proteins for which appropriate therapies have not been developed. Among about 20,000 human proteins, only 3% are currently known to be FDA-approved drug targets, and about 7% of proteins are in principle druggable but are classified as difficult or challenging targets due to lack of success in their drug development so far. Above all, about 3,100 (16%) targets, corresponding to most disease-target proteins, are considered to be “undruggable” because current drug development technology cannot be applied per se (Nature Reviews Drug Discovery, 16, 19-34). Additionally, active site-directed new drugs of the related art are often eventually rendered ineffective due to drug resistance.

Proteolysis-targeting chimera (PROTAC) is a new paradigm to overcome the limitations of conventional drug development technology of the related art. PROTAC utilizes the ubiquitin-proteasome system (UPS), which is an intrinsic protein degradation mechanism in cells to degrade proteins, to induce proteolysis of target proteins that are often hard to be degraded in a natural setting. This “targeted protein degradation (TDP)” technique was first reported in 2001, and is currently receiving great attention from the field, and the related technologies are being actively developed (Cell Res, 26, 484-498). The most revolutionary property of PROTAC is that even various pathological proteins which were previously considered to be difficult targets or undruggable can go through “controlled elimination” by this degradation induction technology. In principle, the PROTAC technology can be applied to all proteins that can be degraded by E3 ligase-mediated ubiquitination mechanisms, and is not limited to specific diseases or target proteins, and thus it has enormous potential to be developed into a platform technology. Indeed, many PROTACs have been actively developed to degrade a plethora of important disease targets, including KRas(G12C), AR(AR-V7), STAT3, Tau, CDK9, BET family, BCR-ABL, BTK, and HDAC family.

However, despite the innovation and great potential value of PROTAC technique, the exact molecular mechanism of targeted protein degradation by this remains elusive. Although it is clear that PROTAC acts by linking E3 ligase to target proteins to form a ternary structure (Drug Discovery Today: Technologies, 31, 15-27), the pattern, form, type, and regulation of ubiquitination by PROTAC have been poorly studied. However, if the poly-Ub or multi-Ub chains that have been formed during the ubiquitination process by PROTAC can be cleaved by deubiquitinase (DUB), PROTAC-mediated substrate degradation would not occur or be downregulated. Therefore, from this perspective, it is highly needed to improve the targeted protein degradation capacity of PROTAC.

### DETAILED DESCRIPTION OF THE DISCLOSURE

### Technical Problem

In order to solve these problems, the inventors of the present application have conducted a series of studies to discover substances to enhance the targeted protein degradation ability by proteolysis-targeting chimera (PROTAC), and as a result, found that the PROTAC function can be significantly enhanced when IU1-series of compounds, which inhibit ubiquitin-specific protease 14 (USP14) among deubiquitinases (DUBs), are treated in combination with PROTAC, and completed the disclosure.

Accordingly, the purpose of the present disclosure is to provide a pharmaceutical composition for preventing or treating cancer, including a USP14 inhibitor and a PROTAC.

Another objective of the present disclosure is to provide a pharmaceutical composition for enhancing the effect of preventing or treating cancer, used with PROTAC in combination, the composition including a USP14 inhibitor as an active ingredient.

Another objective of the present disclosure is to provide a health functional food for preventing or ameliorating cancer, including a USP14 inhibitor and PROTAC.

Another objective of the present disclosure is to provide a method of preventing or treating cancer including administering a USP14 inhibitor and PROTAC to a subject.

Another objective of the present disclosure is to provide use of USP14 inhibitor and PROTAC for the production of drugs for preventing or treating cancer.

However, the technical development to be achieved by the present disclosure is not limited to the issues described above, and other issues not described will be clearly understood by those skilled in the art from the description below.

### Technical Solution to Problem

To achieve these objectives, the present disclosure provides a pharmaceutical composition for preventing or treating cancer, including a ubiquitin-specific protease 14 (USP14) inhibitor and a proteolysis-targeting chimera (PROTAC).

In an embodiment of the present disclosure, the USP14 inhibitor may be an IU1-series compound.

In another embodiment of the present disclosure, the IU1-series compound may be selected from the group consisting of: 1-[1-(4-fluorophenyl)-2,5-dimethylpyrrol-3-yl]-2-pyrrolidin-1-ylethanone; 1-[1-(4-chlorophenyl)-2,5-dimethyl-1H-pyrrol-3-yl]-2-(1-piperidinyl)ethanone; 1-(1-(4-chlorophenyl)-2,5-dimethyl-1H-pyrrol-3-yl)-2-(pyrrolidin-1-yl)ethan-1-one; 4-(3-(2-(4-hydroxypiperidin-1-yl)acetyl)-2,5-dimethyl-1H-pyrrol-1-yl)benzonitrile; 1-(1-(4-chlorophenyl)-5-methyl-1H-pyrazol-4-yl)-2-(piperidin-1-yl)ethan-1-one; 4-(2-methyl-3-(2-(piperidin-1-yl)acetyl)-1H-pyrrolo[3,2-b]pyridin-1-yl)benzonitrile; 1-(4-cyanophenyl)-2-methyl-3-(2-(piperidin-1-yl)acetyl)-1H-pyrrolo[3,2-b]pyridine-5-carbonitrile; 4-(3-(2-((2R)-2-hydroxy-7-azabicyclo[2.2.1]heptan-7-yl)acetyl)-2-methyl-5-(3-(tetrahydro-2H-pyran-4-yl)propyl)-1H-pyrrol-1- yl)benzonitrile; (E)-2-(7-azabicyclo[2.2.1]heptan-7-yl)-1-(1-(4-chlorophenyl)-2-methyl-5-(4-(methylsulfonyl)but-1-en-1-yl)-1H-pyrrol-3- yl)ethan-1-one; 4-(3-(2-((1r,3r,5r,7r-2-azaadamantan-2-yl)acetyl)-2,5-dimethyl-1H-pyrrol-1-yl)benzonitrile; and 4-(5-((E)-3-(1,3-dioxoisoindolin-2-yl)prop-1-en-1-yl)-3-(2-((2R)-2-hydroxy-7-azabicyclo[2.2.1]heptan-7-yl)acetyl)-2-methyl-1H-pyrrol-1-yl)benzonitrile.

In another embodiment of the present disclosure, the cancer may be brain tumor, head and neck cancer, breast cancer, lung cancer, esophageal cancer, stomach cancer, duodenal cancer, appendix cancer, colon cancer, rectal cancer, liver cancer, pancreatic cancer, gallbladder cancer, bile duct cancer, anal cancer, renal cancer, ureteral cancer, bladder cancer, prostate cancer, penile cancer, testicular cancer, uterine cancer, ovarian cancer, vulvar cancer, vaginal cancer, or skin cancer.

In another embodiment of the present disclosure, the PROTAC may target a protein selected from the group consisting of cyclin-dependent kinase 9 (CDK9)/CDK family, bromodomain-containing protein 4 (BRD4)/bromodomain and extra-terminal (BET) family, Src homology region 2 domain-containing phosphatase-2 (SHP-2), androgen receptor (AR), estrogen receptor (ER), retinoic acid receptor (RAR), estrogen-related receptor alpha (ERRα), Bruton's tyrosine kinase (BTK), anaplastic lymphoma kinase (ALK), receptor-interacting serine/threonine-protein kinase (RIPK), MET proto-oncogene/receptor tyrosine kinase (c-Met), focal adhesion kinase (FAK), interleukin-1 receptor-associated kinase 4 (IRAK4), p38 mitogen-activated protein kinases (p38 MAPK), serum and glucocorticoid-regulated kinase (SGK), TANK-binding kinase 1 (TBK1), Kirsten rat sarcoma virus protein (KRAS), B-Raf proto-oncogene serine/threonine kinase (B-Raf), β-catenin, FK506 binding protein (FKBP), indoleamine 2,3-dioxygenase (IDO), programmed death protein 1/programmed death-ligand 1 (PD-1/PD-L1), poly[ADP-ribose]polymerase 1 (PARP1), polycomb repressive complex 2 (PRC2), epidermal growth factor receptor (EGFR), NAD-dependent deacetylase sirtuin 2 (Sirt2), human epidermal growth factor receptor 2 (HER2/ERBB2), fibroblast growth factor receptor substrate 2 (FRS2), B-cell lymphoma-extra large (BCL-XL), SWI/SNF related matrix associated actin dependent regulator of chromatin subfamily A (SMARCA), human double minute 2 homolog (HDM2), histone deacetylase (HDAC) family, breakpoint cluster region protein- tyrosine-protein kinase ABL1 (BCR-ABL), modulator of VRAC current 1 (MCL1), Fms related receptor tyrosine kinase 3 (FLT-3), transcription factor STAT3 (STAT3), Myc family, and Brg/Brahma-associated factors (BAF complex).

In another embodiment of the present disclosure, the protein degradation effect may be obtained through the E3 ligase, on which the PROTAC operates, selected from the group consisting of Cereblon (CRBN), Von Hippel-Lindau tumor suppressor (VHL), cellular inhibitor of apoptosis protein (cIAP), Kelch-like ECH-associated protein 1 (Keap1), human double minute 2 homolog (HDM2), beta-transducin repeat containing E3 ubiquitin protein ligase (βTRCP), carboxyl terminus of HSC70-interacting protein (CHIP), and ubiquitin like with PHD and ring finger domains 1 (UHRF1).

In another embodiment of the present disclosure, the USP14 inhibitor may enhance the protein degradation effect of PROTAC.

The present disclosure also provides a pharmaceutical composition for enhancing the effect of preventing or treating cancer, when used with PROTAC in combination, including a USP14 inhibitor as an active ingredient.

In an embodiment of the present disclosure, the USP14 inhibitor may be an IU1-series compound.

In another embodiment of the present disclosure, the IU1-series compound may be selected from the group consisting of: 1-[1-(4-fluorophenyl)-2,5-dimethylpyrrol-3-yl]-2-pyrrolidin-1-ylethanone; 1-[1-(4-chlorophenyl)-2,5-dimethyl-1H-pyrrol-3-yl]-2-(1-piperidinypethanone; 1-(1-(4-chlorophenyl)-2,5-dimethyl-1H-pyrrol-3-yl)-2-(pyrrolidin-1-yl)ethan-1-one; 4-(3-(2-(4-hydroxypiperidin-1-yl)acetyl)-2,5-dimethyl-1H-pyrrol-1-yl)benzonitrile; 1-(1-(4-chlorophenyl)-5-methyl-1H-pyrazol-4-yl)-2-(piperidin-1-yl)ethan-1-one; 4-(2- 4-(2-methyl-3-(2-(piperidin-1 -yl)acetyl)-1H-pyrrolo[3,2-b]pyridin-1-yl)benzonitrile; 1-(4-cyanophenyl)-2-methyl-3-(2-(piperidin-1- yl)acetyl)-1H-pyrrolo[3,2-b]pyridine-5-carbonitrile; 4-(3-(2-((2R)-2-hydroxy-7-azabicyclo[2.2.1]heptan-7-yl)acetyl)-2-methyl-5-(3- (tetrahydro-2H-pyran-4-yl)propyl)-1H-pyrrol-1-yl)benzonitrile; (E)-2-(7-azabicyclo[2.2.1]heptan-7-yl)-1-(1-(4-chlorophenyl)-2-methyl-5-(4-(methylsulfonyl)but-1-en-1-yl)-1H-pyrrol-3-yl)ethan-1-one; 4-(3-(2-((1 r,3r,5r,7r-2-azaadamantan-2-yl)acetyl)-2,5-dimethyl-1H-pyrrol-1-yl)benzonitrile; and 4-(5-((E)-3-(1,3-dioxoisoindolin-2-yl)prop-1-en-1-yl)-3-(2-((2R)-2-hydroxy-7-azabicyclo[2.2.1]heptan-7-yl)acetyl)-2-methyl-1H-pyrrol-1-yl)benzonitrile.

In another embodiment of the present disclosure, the cancer may be brain tumor, head and neck cancer, breast cancer, lung cancer, esophageal cancer, stomach cancer, duodenal cancer, appendix cancer, colon cancer, rectal cancer, liver cancer, pancreatic cancer, gallbladder cancer, bile duct cancer, anal cancer, renal cancer, ureteral cancer, bladder cancer, prostate cancer, penile cancer, testicular cancer, uterine cancer, ovarian cancer, vulvar cancer, vaginal cancer, or skin cancer.

In another embodiment of the present disclosure, the PROTAC may target a protein selected from the group consisting of cyclin-dependent kinase 9 (CDK9)/CDK family, bromodomain-containing protein 4 (BRD4)/bromodomain and extra-terminal (BET) family, Src homology region 2 domain-containing phosphatase-2 (SHP-2), androgen receptor (AR), estrogen receptor (ER), retinoic acid receptor (RAR), estrogen-related receptor alpha (ERRα), Bruton's tyrosine kinase (BTK), anaplastic lymphoma kinase (ALK), receptor-interacting serine/threonine-protein kinase (RIPK), MET proto-oncogene/receptor tyrosine kinase (c-Met), focal adhesion kinase (FAK), interleukin-1 receptor-associated kinase 4 (IRAK4), p38 mitogen-activated protein kinases (p38 MAPK), serum and glucocorticoid-regulated kinase (SGK), TANK-binding kinase 1 (TBK1), Kirsten rat sarcoma virus protein (KRAS), B-Raf proto-oncogene serine/threonine kinase (B-Raf), β-catenin, FK506 binding protein (FKBP), indoleamine 2,3-dioxygenase (IDO), programmed death protein 1/programmed death-ligand 1 (PD-1/PD-L1), poly[ADP-ribose]polymerase 1 (PARP1), polycomb repressive complex 2 (PRC2), epidermal growth factor receptor (EGFR), NAD-dependent deacetylase sirtuin 2 (Sirt2), human epidermal growth factor receptor 2 (HER2/ERBB2), fibroblast growth factor receptor substrate 2 (FRS2), B-cell lymphoma-extra large (BCL-XL), SWI/SNF related matrix associated actin dependent regulator of chromatin subfamily A (SMARCA), human double minute 2 homolog (HDM2), histone deacetylase (HDAC) family, breakpoint cluster region protein- tyrosine-protein kinase ABL1 (BCR-ABL), modulator of VRAC current 1 (MCL1), Fms related receptor tyrosine kinase 3 (FLT-3), transcription factor STAT3 (STAT3), Myc family, and Brg/Brahma-associated factors (BAF complex).

In another embodiment of the present disclosure, the protein degradation effect may be obtained through the E3 ligase, on which the PROTAC operates, selected from the group consisting of cereblon (CRBN), Von Hippel-Lindau tumor suppressor (VHL), cellular inhibitor of apoptosis protein (cIAP), Kelch-like ECH-associated protein 1 (Keap1), human double minute 2 homolog (HDM2), beta-transducin repeat containing E3 ubiquitin protein ligase (βTRCP), carboxyl terminus of HSC70-interacting protein (CHIP), and ubiquitin like with PHD and ring finger domains 1 (UHRF1).

In another embodiment of the present disclosure, the USP14 inhibitor may enhance the protein degradation effect of PROTAC.

In another embodiment of the present disclosure, the use in combination may be administering simultaneously, separately, or sequentially with PROTAC.

The present disclosure also provides a health functional food for preventing or ameliorating cancer, including a USP14 inhibitor and PROTAC.

The present disclosure also provides a method of preventing or treating cancer, including administering a USP14 inhibitor and PROTAC to a subject.

The present disclosure also provides use of USP14 inhibitors and PROTAC for the production of drugs for preventing or treating cancer.

### Advantageous Effects of Disclosure

According to the present disclosure, when the IU1-series compound, which is an inhibitor of USP14, is administered in combination with proteolysis-targeting chimera (PROTAC), the targeted protein degradation and anticancer effect of PROTAC are enhanced. Accordingly, it is expected that the IU1 -series compounds of the present disclosure may be useful in the prevention and treatment of cancer in the future.

However, the effects of the present disclosure are not limited to those described above, and should be understood to include all the effects that can be inferred from the features of the disclosure described in the detailed description or claims of the present disclosure.

### BRIEF DESCRIPTION OF DRAWINGS

FIG. 1 shows a schematic diagram showing the targeted protein degradation process by proteolysis-targeting chimera (PROTAC).

FIG. 2 shows a schematic diagram showing a method of enhancing the targeted protein degradation capability of PROTAC based on the deubiquitinase inhibitor proposed according to the present disclosure.

FIG. 3 shows the targeted protein degradation effect of CDK9 PROTAC: FIG. 3A shows the structure and mode of action of PROTAC targeting CDK9; and FIG. 3B shows the results of the degradation of CDK9 in LNCaP, MB231 and HeLa cells when treated with CDK9 PROTAC at different concentrations.

FIG. 4 shows the effect of the IU1 compound on enhancing the protein degradation capability of CDK9 PROTAC: FIG. 4A shows a comparison of the degradation level of the target protein (CDK9) in the experimental group treated with CDK9 PROTAC alone versus in the experimental group treated with PROTAC and IU1 compound in combination; FIG. 4B shows whether the CDK9 degradation also occurs when treated with the IU1 compound alone; and FIG. 4C shows the results of the degradation effect of CDK9 PROTAC in the presence of a proteasome inhibitor (MG132) or an autophagy inhibitor (Baf) to determine the mechanism of the IU1 compound in enhancing the protein degradation capability of CDK9 PROTAC.

FIGS. 5A and 5B show the results of confirming the effect of the IU1 compound on enhancing the protein degradation capability of PROTAC in a CDK9 PROTAC concentration-dependent manner after setting the IU1 compound at each different concentration in HeLa cells.

FIGS. 6A and 6B show the results of confirming the effect of the IU1 compound on enhancing the protein degradation capability of PROTAC in an IU1 concentration-dependent manner after setting CDK9 PROTAC at each different concentration in HeLa cells.

FIG. 7 shows the results of identifying the level of target protein (CDK9) degradation, measured as the DC₅₀ values, by the combined treatment of IU1 compound and CDK9 PROTAC: FIG. 7A and FIG. 7B show the degradation levels of target proteins in HeLa cells when treated with or without the IU1 compound at different concentrations, while increasing the treatment concentrations of PROTAC, and FIG. 7C shows the results of quantifying the results of FIGS. 7A and 7B to determine the DC₅₀ values depending on the concentrations of the treatment materials.

FIG. 8 shows the results of testing whether IU1 compound can enhance the anticancer effect of PROTAC, and the results of testing the inhibitory effect of PROTAC on cancer cell growth with or without the treatment of IU1 compound.

FIG. 9 shows the results of treating LNCaP cell line, which is a prostate cancer cell line, with PROTAC alone or in combination with the IU1 compound and then identifying the degradation level of target protein in order to determine whether IU1 of the present disclosure can enhance the anticancer effect of PROTAC in cancer types other than HeLa cells, which is a cervical cancer cell line.

FIGS. 10A and 10B show the results of confirming the effect of the IU1 compound on enhancing the protein degradation capability of PROTAC in a CDK9 PROTAC concentration-dependent manner after setting the IU1 compound at each different concentration in LNCaP cells.

FIG. 11 shows the results of examining the degradation levels of target protein when treated with BRD4-targeting PROTAC alone and in combination with an IU1 compound in order to determine whether co-administration of the IU1 compound of the present disclosure can enhance the targeted protein degradation capability of PROTAC other than CDK9 PROTAC.

FIG. 12A and FIG. 12B show the results of confirming the degradation levels of target proteins when treated with the SHP-2-targeting PROTAC alone at a concentration similar to that of CDK9 PROTAC and when treated in combination with the IU1 compound, to determine whether co-administration of the IU1 compound of the present disclosure with PROTAC other than CDK9 PROTAC and BRD4 PROTAC in HeLa cells can enhance the target protein degradation by PROTAC.

FIG. 13 shows the degradation level of the target protein (CDK9), measured as the DC₅₀ value, when treated with IU1-47, which is a more potent IU1 derivative, and CDK9 PROTAC in combination in order to determine whether other IU1-series compounds also exert the degradation enhancing capability with PROTAC: FIGS. 13A and 13B show the degradation levels of target proteins in HeLa cells with or without the treatment of the IU1-47 compound at different concentrations, while increasing the treatment concentrations of PROTAC; and FIG. 13C shows the quantifying results of FIGS. 13A and 13B to determine the DC₅₀ values depending on the concentrations of the treatment materials.

FIG. 14 shows the effect of the IU1-47 compound on enhancing the protein degradation capability of CDK9 PROTAC in an IU1-47-concentration dependent manner after setting CDK9 PROTAC at each different concentration in HeLa cells.

FIG. 15 shows the results of confirming the effect of the IU1 compound on enhancing the protein degradation capability of CDK9 PROTAC in a time-dependent manner when treated with CDK9 PROTAC alone or in combination with IU1 in HeLa cells.

FIG. 16 shows the results of comparing the effect of IU1-47 compound to that of PR-619 compound, which is the non-specific deubiquitinase inhibitor (pan DUB inhibitor), on enhancing the protein degradation capability of CDK9 PROTAC after setting PR-619 compound at each different concentration in HeLa cells.

FIG. 17 shows the results of comparing the effect of IU1-47 compound to that of P50429 compound, which is a ubiquitin-specific protease 7/47 dual inhibitor, on enhancing the protein degradation capability of CDK9 PROTAC after setting P50429 compound at each different concentration in HeLa cells.

FIG. 18 shows the results of a cell migration assay to determine the change in cancer cell migration using HeLa cells by enhancement of CDK9 protein degradation when CDK9 PROTAC and IU1-47 were used separately or in combination.

FIG. 19 shows the results of a colony formation assay to determine the change in the ability of cancer cells to form colonies using HeLa cells by enhancement of CDK9 protein degradation when treated with different concentrations of CDK9 PROTAC alone or in combination with the IU1-47 compounds.

### MODE OF DISCLOSURE

The inventors confirmed that the protein degradation function of PROTAC was enhanced when PROTAC is used in combination with an IU1-series compound that inhibits ubiquitin-specific protease 14 (USP14) among deubiquitinases that suppress the protein degradation function of PROTAC, and completed the present disclosure.

Accordingly, the present disclosure provides a pharmaceutical composition for preventing or treating cancer, including a USP14 inhibitor and PROTAC.

The term “USP14” used in the present disclosure is one of about 100 deubiquitinases (DUBs) in the human proteome, which are the only family of enzymes that can reverse ubiquitination, can be associated with the proteasome, and acts specifically on multiple ubiquitin chains.

The USP14 inhibitor used herein may be an IU1-series compound, and the IU1-series compound may be, not limited thereto, selected from the group consisting of: 1-[1-(4-fluorophenyl)-2,5-dimethylpyrrol-3-yl]-2-pyrrolidin-1-ylethanone; 1-[1(4-chlorophenyl)-2,5-dimethyl-1H-pyrrol-3-yl]-2-(1-piperidinypethanone; 1-(1-(4-chlorophenyl)-2,5-dimethyl-1H-pyrrol-3-yl)-2-(pyrrolidin-1-yl)ethan-1-one; 4-(3-(2-(4-hydroxypiperidin-1-yl)acetyl)-2,5-dimethyl-1H-pyrrol-1-yl)benzonitrile; 1-(1-(4-chlorophenyl)-5-methyl-1H-pyrazol-4-yl)-2-(piperidin-1-yl)ethan-1-one; 4-(2-methyl-3-(2-(piperidin-1-yl)acetyl)-1 H-pyrrolo[3,2-b]pyridin-1-yl)benzonitrile; 1-(4-cyanophenyl)-2-methyl-3-(2-(piperidin-1-yl)acetyl)-1H-pyrrolo[3,2-b]pyridine-5-carbonitrile; 4-(3-(2-((2R)-2-hydroxy-7-azabicyclo[2.2.1]heptan-7-yl)acetyl)-2-methyl-5-(3-(tetrahydro-2H-pyran-4-yl)propyl)-1H-pyrrol-1- yl)benzonitrile; (E)-2-(7-azabicyclo[2.2.1]heptan-7-yl)-1-(1-(4-chlorophenyl)-2-methyl-5-(4-(methylsulfonyl)but-1-en-1-yl)-1H-pyrrol-3- yl)ethan-1-one; 4-(3-(2-((1r,3r,5r,7r)-2-azaadamantan-2-yl)acetyl)-2,5-dimethyl-1H-pyrrol-1-yl)benzonitrile; and 4-(5-((E)-3-(1,3-dioxoisoindolin-2-yl)prop-1-en-1-yl)-3-(2-((2R)-2-hydroxy-7-azabicyclo[2.2.1]heptan-7-yl)acetyl)-2-methyl-1H-pyrrol-1 -yl)benzonitrile or derivatives derived therefrom, and may have the structure of

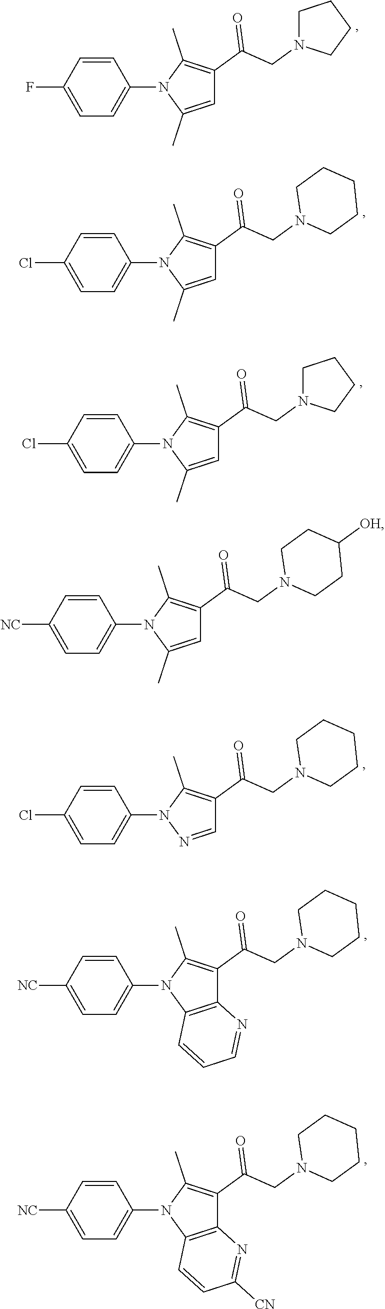

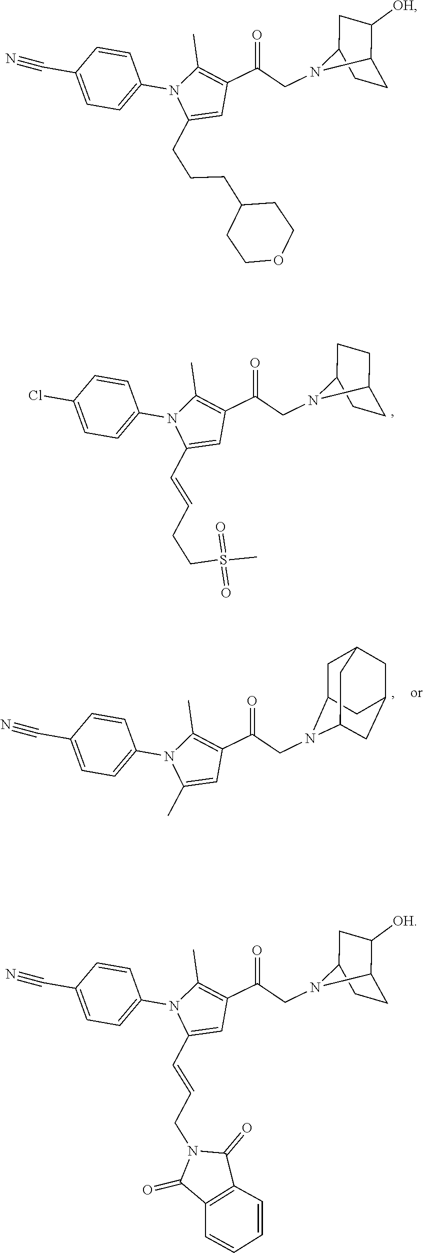 [SMILES: Cc1c(C(=O)CN2C3CCC2C(O)C3)cc(CCCC2CCOCC2)n1-c1ccc(C#N)cc1 ; Cc1c(C(=O)CN2C3CCC2CC3)cc(/C=C/CCS(C)(=O)=O)n1-c1ccc(Cl)cc1 ; Cc1cc(C(=O)CN2C3CC4CC(C3)CC2C4)c(C)n1-c1ccc(C#N)cc1 ; Cc1c(C(=O)CN2C3CCC2C(O)C3)cc(/C=C/CN2C(=O)c3ccccc3C2=O)n1-c1ccc(C#N)cc1]

In an embodiment, the USP14 inhibitor may be selected from 1[1-(4-fluorophenyl)-2,5-dimethylpyrrol-3-yl]-2-pyrrolidin-1-ylethanone, 1-[1-(4-chlorophenyl)-2,5-dimethyl-1H-pyrrol-3-yl]-2-(1-piperidinyl)ethanone or analogs derived therefrom, and may have the structure of

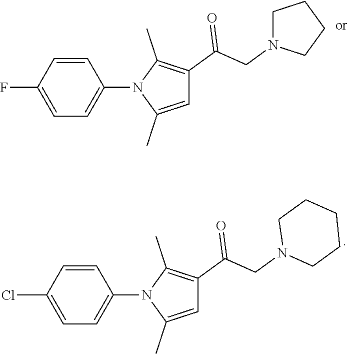 [SMILES: Cc1cc(C(=O)CN2CCCC2)c(C)n1-c1ccc(F)cc1 ; Cc1cc(C(=O)CN2CCCCC2)c(C)n1-c1ccc(Cl)cc1]

In an embodiment, the USP14 inhibitor may be IU1 or IU1-47.

In an embodiment, the USP14 inhibitor may enhance the protein degradation effect of PROTAC.

The proteasome is one of the components of the ubiquitin-proteasome system (UPS), which is a system that degrades intracellular proteins, and serves to degrade proteins by breaking the peptide bonds of ubiquitin-labeled substrate proteins.

The term “protein” as used in the present disclosure may be used interchangeably with “polypeptide” and refers to a polymer of amino acid residues. The protein corresponds to amino acid polymers that are artificial chemical mimics of naturally occurring amino acids, naturally occurring amino acid polymers, those containing modified residues, and non-naturally occurring amino acid polymers, to which one or more amino acid residues correspond.

The term “proteolysis-targeting chimera (PROTAC)” used in the present disclosure is a platform technology consisting of an E3 ligase binding module-linker-target protein binding module, which degrades disease-causing target proteins in vivo by inducing ubiquitination. PROTAC may inhibit, through degradation, most of disease-causing proteins that are difficult to be inhibited by active site-directed drugs derived from conventional drug development methods.

PROTAC used herein may target a protein selected from the group consisting of cyclin-dependent kinase 9 (CDK9)/CDK family, bromodomain-containing protein 4 (BRD4)/bromodomain and extra-terminal (BET) family, Src homology region 2 domain-containing phosphatase-2 (SHP-2), androgen receptor (AR), estrogen receptor (ER), retinoic acid receptor (RAR), estrogen-related receptor alpha (ERRα), Bruton's tyrosine kinase (BTK), anaplastic lymphoma kinase (ALK), receptor-interacting serine/threonine-protein kinase (RIPK), MET proto-oncogene/receptor tyrosine kinase (c-Met), focal adhesion kinase (FAK), interleukin-1 receptor-associated kinase 4 (IRAK4), p38 mitogen-activated protein kinases (p38 MAPK), serum and glucocorticoid-regulated kinase (SGK), TANK-binding kinase 1 (TBK1), Kirsten rat sarcoma virus protein (KRAS), B-Raf proto-oncogene serine/threonine kinase (B-Raf), β-catenin, FK506 binding protein (FKBP), indoleamine 2,3-dioxygenase (IDO), programmed death protein 1/programmed death-ligand 1 (PD-1/PD-L1), poly[ADP-ribose]polymerase 1 (PARP1), polycomb repressive complex 2 (PRC2), epidermal growth factor receptor (EGFR), NAD-dependent deacetylase sirtuin 2 (Sirt2), human epidermal growth factor receptor 2 (HER2/ERBB2), fibroblast growth factor receptor substrate 2 (FRS2), B-cell lymphoma-extra large (BCL-XL), SWI/SNF related matrix associated actin dependent regulator of chromatin subfamily A (SMARCA), human double minute 2 homolog (HDM2), histone deacetylase (HDAC) family, breakpoint cluster region protein- tyrosine-protein kinase ABL1 (BCR-ABL), modulator of VRAC current 1 (MCL1), Fms related receptor tyrosine kinase 3 (FLT-3), transcription factor STAT3 (STAT3), Myc family, and Brg/Brahma-associated factors (BAF complex). In an embodiment, PROTAC may target a protein selected from the group consisting of cyclin-dependent kinase 9 (CDK9)/CDK family, bromodomain-containing protein 4 (BRD4)/bromodomain and extra-terminal (BET) family, and Src homology region 2 domain-containing phosphatase-2 (SHP-2).

According to the present disclosure, the protein degradation effect may occur through E3 ligase, on which the PROTAC operates, selected from the group consisting of cereblon (CRBN), Von Hippel-Lindau tumor suppressor (VHL), cellular inhibitor of apoptosis protein (cIAP), Kelch-like ECH-associated protein 1 (Keap1), human double minute 2 homolog (HDM2), beta-transducin repeat containing E3 ubiquitin protein ligase (βTRCP), carboxyl terminus of HSC70-interacting protein (CHIP), and ubiquitin like with PHD and ring finger domains 1 (UHRF1). In an embodiment, the protein degradation effect may occur through E3 ligase selected from the group consisting of cereblon (CRBN) and Von Hippel-Lindau tumor suppressor (VHL).

The term “cancer” used in the present disclosure refers to a general term for diseases caused by cells with aggressive characteristics in which cells divide and grow ignoring normal growth limits, invasive characteristics that penetrate into surrounding tissues, and metastatic characteristics that spread to other parts of the body.

Regarding the present disclosure, the type of cancer is not particularly limited and includes brain tumor, head and neck cancer, breast cancer, lung cancer, esophageal cancer, stomach cancer, duodenal cancer, appendix cancer, colon cancer, rectal cancer, liver cancer, pancreatic cancer, gallbladder cancer, bile duct cancer, anus cancer, renal cancer, ureteral cancer, bladder cancer, prostate cancer, penile cancer, testicular cancer, uterine cancer, ovarian cancer, vulvar cancer, vaginal cancer, or skin cancer. In an embodiment of the present disclosure, the anticancer effects of co-administration of IU1-series compounds and PROTAC in uterine and prostate cancer cell lines have been confirmed, but are not limited to.

The term “prevention” as used in the present disclosure, refers to any act of inhibiting cancer or delaying the onset of cancer by administration of a pharmaceutical composition according to the present disclosure.

The term “treatment” as used in the present disclosure refers to any act by which the symptoms of a cancer are ameliorated or beneficially altered by the administration of a pharmaceutical composition according to the present disclosure.

Through specific examples, the present inventors confirmed that IU1-series compounds according to the present disclosure can enhance the anticancer effect of PROTAC.

In an embodiment of the present disclosure, it was found that the combined treatment of IU1, which is an IU1-series compound, and PROTAC, which targets CDK9, significantly enhanced the CDK9 proteolytic effect of PROTAC, even though no CDK9 protein degradation effect was observed when treated with IU1 alone, and that this effect was dependent on the concentration and treatment time of both IU1 and PROTAC (see Examples 2 and 7).

In another example of the present disclosure, it was confirmed that the enhancement of the CDK9 protein degradation by co-treatment of IU1 and CDK9 PROTAC as shown in Example 2 was not simply limited to the protein degradation enhancing effect, but actually inhibited the growth of cancer cells. (See Example 3).

In another embodiment of the present disclosure, to confirm that the targeted protein degradation and anticancer effect by the combination of IU1 and PROTAC of the present disclosure is not only effective on HeLa cells, which is a uterine cancer cell line, but can also be applied to general cancer cells, it was confirmed that when LNCaP cells, which is a prostate cancer cell line, were co-treated with IU1 and PROTAC, the target protein (CDK9) degradation capability by PROTAC was significantly improved, as was the case in HeLa cells (see Example 4).

In another embodiment of the present disclosure, to identify whether the protein degradation capability could be enhanced for PROTACs other than PROTAC targeting CDK9, it was confirmed whether the BRD4 protein degradation by BRD4 PROTAC, which is an E3 ligase VHL-based PROTAC, was enhanced. As a result, it was confirmed that when IU1 was treated in combination with BRD4 PROTAC, the BRD4 protein degradation was enhanced in a concentration-dependent manner (see Example 5).

In addition, as a result of identifying whether the SHP-2 protein degradation can be enhanced by SHP-2 PROTAC, which is another VHL-based PROTAC other than BRD4 PROTAC, it was confirmed that when IU1 was treated in combination with SHP-2 PROTAC, the SHP-2 protein degradation was enhanced in a SHP-2 PROTAC concentration-dependent manner (see Example 5).

In another embodiment of the present disclosure, when IU1-47, one of the IU1-series compounds of the present disclosure, is used, the targeted protein degradation capability of PROTAC was improved, similar to the case of treating IU1 in combination with PROTAC, and rather it was confirmed that when treated with IU1-47, the targeted protein degradation capability of PROTAC was more increased compared to when IU1 was treated in combination with PROTAC (see Example 6).

In another embodiment of the present disclosure, to determine whether deubiquitinase inhibitors other than the IU1 series have the effect on enhancing the protein degradation capability of PROTAC, the effect of the PR-619 compound, which is a non-specific deubiquitinase inhibitor (pan DUB inhibitor), on enhancing the protein degradation capability of the CDK9 PROTAC in HeLa cells was compared with that of IU1-47. As a result, it was confirmed that IU1-47 significantly enhanced the degradation effect of PROTAC, while PR-619 did not enhance the degradation effect of PROTAC at all treatment concentrations (see Example 8).

In addition, the enhancement of protein degradation capability of PROTAC by treatment with USP7/47 dual inhibitor (hereinafter referred to as P50429), which is a dual deubiquitinase inhibitor of USP7 and USP47, and CDK9 PROTAC in combination, was compared with that of IU1-47. As a result, it was confirmed that IU1-47 (20 μM) significantly enhanced the degradation effect of PROTAC, while P50429 did not enhance the degradation effect of PROTAC (see Example 8).

In another embodiment of the present disclosure, it was confirmed that the enhancement of target protein degradation by co-treatment of IU1-series compound and PROTAC as shown in the previous Examples actually suppressed the properties which are intimately associated with cancer cell metastasis and tumorigenesis. As the results, compared to the IU1-47 compound or CDK9 treatment alone, the treatment of IU1-47 compound and CDK9 PROTAC in combination more significantly inhibited cancer cell migration as well as the colony formation ability in HeLa cells (see Example 9).

Through these results, the inventors have confirmed that the IU1-series compounds, which are USP14 inhibitors of the present disclosure, when administered in combination with PROTAC, can significantly enhance the degradation effect and anticancer effect of PROTAC through targeted protein degradation, and thus can be widely used in the field of cancer treatment.

Another aspect of the present disclosure provides a pharmaceutical composition for enhancing the effect of preventing or treating cancer, used with PROTAC in combination, the composition including a USP14 inhibitor as an active ingredient.

The “PROTAC,” “cancer,” “prevention,” “treatment,” “USP14 inhibitor,” etc. may be within the described scope.

The term “enhancing” in the context of the present disclosure refers to inhibiting cancer, delaying the onset of cancer, or significantly ameliorating symptoms caused by cancer in the case where a pharmaceutical composition for the prevention or treatment of cancer according to the present disclosure is administered in combination with PROTAC to a greater extent than when PROTAC is administered alone.

The use in combination may be administering simultaneously, separately, or sequentially with PROTAC.

Regarding the present disclosure, when the pharmaceutical composition for enhancing the effect of preventing or treating cancer is co-administered with PROTAC, the degradation effect on the targeted protein and the anticancer effect of PROTAC are significantly improved compared to when PROTAC is administered alone.

The term “ administration ” in the present disclosure refers to the introduction of a pharmaceutical composition of the present disclosure into a subject by any suitable method, and the route of administration may vary from oral to parenteral, as long as the target tissue can be reached. Examples of parenteral routes of administration include a subcutaneous route, an intraperitoneal route, an intrapulmonary route, an intranasal route, an intramuscular route, an intravenous route, or an intraarterial route.

The pharmaceutical composition of the present disclosure may further include appropriate carriers, excipients, or diluents which are commonly used in the preparation of pharmaceutical compositions. A composition containing a pharmaceutically acceptable carrier may be in various oral or parenteral dosage forms. When formulated, diluents or excipients, such as fillers, extenders, binders, wetting agents, disintegrants, and surfactants, which are commonly used, may be used. Formulations for oral administration may include tablets, pills, powders, granules, capsules, suspensions, oral solutions, emulsions, syrups, sterilized aqueous solutions, non-aqueous solvents, freeze-dried preparations, etc. These formulations may be prepared by mixing one or more compounds with at least one excipient, for example, starch, calcium carbonate, sucrose or lactose, gelatin, etc.

Additionally, in addition to simple excipients, lubricants, such as magnesium stearate, talc, etc., may also be used. Liquid formulations for oral administration include suspensions, oral solutions, emulsions, and syrups. In addition to the commonly used simple diluents such as water and liquid paraffin, various excipients, such as wetting agents, sweeteners, fragrances, and preservatives, may be included. Formulations for parenteral administration may include sterilized aqueous solutions, non-aqueous solvents, suspensions, emulsions, freeze-dried preparations, and suppositories. Non-aqueous solvents and suspension solvents may be selected from propylene glycol, polyethylene glycol, and vegetable oil such as olive oil. As a base for suppositories, witepsol, macrogol, tween 61, cacao, laurel, glycerogelatin, etc. may be used.

In an embodiment, the pharmaceutical composition of the present disclosure may be administered in a pharmaceutically effective amount. The term “pharmaceutically effective amount” in the present disclosure refers to an amount sufficient to suppress or alleviate cancer with a reasonable benefit/risk ratio applicable to medical use, and the effective dose level may be determined by the type and severity of the subject, age, weight, diet, gender, drug activity, sensitivity to the drug, time of administration, route of administration and excretion rate, duration of treatment, factors including concurrently used drugs, and other factors well known in the medical field.

The pharmaceutical composition of the present disclosure may be administered once a day, several times, once every other day, or once a week. Specifically, the pharmaceutical composition may be administered at 0.001 mg/kg/day to 1000 mg/kg/day, for example, at 0.1 mg/kg/day to 100 mg/kg/day.

The pharmaceutical composition of the present disclosure may be administered in combination with a known composition or other pharmaceutical composition that has a cancer prevention or treatment effect, and may be administered simultaneously, separately, or sequentially, and may be administered in single or multiple doses. Considering all of these factors, it is important to administer an amount that can achieve the maximum effect with the minimum amount without side effects, and this could be easily determined by a person skilled in the art.

Another aspect of the present disclosure provides a health functional food for preventing or ameliorating cancer, including a USP14 inhibitor and PROTAC.

The terms “USP14 inhibitor,” “PROTAC,” “cancer,” “prevention,” etc. may be within the described scope.

Regarding the present disclosure, when the health functional food includes a USP14 inhibitor and PROTAC, the cancer prevention or amelioration effect is superior to when including PROTAC alone.

The term “amelioration” may refer to any act that at least reduces a parameter related to the condition being treated, for example, the severity of symptoms. In this regard, the health functional food may be used simultaneously with or independently of a therapeutic agent for the prevention or amelioration of cancer, either before or after the onset of the disease.

Regarding the health functional food, the active ingredient may be added directly to the food or used together with other foods or other food ingredients, and may be used appropriately according to methods of the related art. The mixing amount of the active ingredient may be appropriately determined depending on the purpose of use (prevention or amelioration). In general, when manufacturing a food or beverage, the health functional food may be added in an amount of about 15% by weight or less, for example, about 10% by weight or less, based on the raw materials. However, in the case of long-term intake for the purpose of health and hygiene or health control, the amount may be equal to or lower than the ranges described above.

The health functional food may be prepared in a formulation selected from the group consisting of tablets, pills, powders, granules, powders, capsules, and liquid formulations while further including one or more selected from carriers, diluents, excipients, and additives. Foods, to which compounds according to an aspect can be added, may include various foods, powders, granules, tablets, capsules, syrups, beverages, gum, tea, vitamin complexes, health functional foods, etc.

Specific examples of the carriers, excipients, diluents, and additives include one or more selected from a group consisting of lactose, dextrose, sucrose, sorbitol, mannitol, erythritol, starch, gum acacia, calcium phosphate, alginate, gelatin, calcium phosphate, calcium silicate, microcrystalline cellulose, polyvinylpyrrolidone, cellulose, methylcellulose, water, sugar syrup, methyl hydroxy benzoate, propylhydroxy benzoate, talc, magnesium stearate, and mineral oil.

In addition to including the active ingredient, the health functional food may include other ingredients as essential ingredients without any particular restrictions. For example, like regular beverages, it may include various flavoring agents or natural carbohydrates as additional ingredients. Examples of the natural carbohydrates include common sugars, for example: monosaccharides such as glucose, fructose, etc.; disaccharides such as maltose, sucrose, etc.; and polysaccharides, such as dextrin and cyclodextrin, and sugar alcohols such as xylitol, sorbitol, and erythritol. As flavoring agents other than those described above, natural flavoring agents (thaumatin, stevia extract (e.g., rebaudioside A, glycyrrhizin, etc.)) and synthetic flavoring agents (saccharin, aspartame, etc.) may be advantageously used. The ratio of the natural carbohydrates may be appropriately determined by the selection of a person skilled in the art.

In addition to the materials described above, the health functional food of the present disclosure may include various nutrients, vitamins, minerals (electrolytes), flavoring agents such as synthetic and natural flavors, colorants and thickening agents (cheese, chocolate, etc.), pectic acid and salts thereof, alginic acid and salts thereof, organic acids, protective colloidal thickeners, pH adjusters, stabilizers, preservatives, glycerin, alcohol, carbonating agents used in carbonated beverages, etc. These components may be used independently or in combination, and the proportions of these additives may also be appropriately selected by a person skilled in the art.

The health functional food may be provided in combination with a conventionally known health functional food for preventing or ameliorating cancer or a newly developed health functional food for preventing or ameliorating cancer.

When the health functional food further includes a health functional food for preventing or ameliorating cancer, it is important to mix the mixture in an amount that can obtain the maximum effect with the minimum amount without side effects, which may be easily determined by a person skilled in the art.

In another aspect of the present disclosure, the present disclosure provides a method of preventing or treating cancer including administering a USP14 inhibitor and PROTAC to a subject.

The terms “USP14 inhibitor,” “PROTAC,” “administration,” “cancer,” “prevention,” “treatment,” etc. may be within the described scope.

Regarding the method of the present disclosure, when a USP14 inhibitor and PROTAC are co-administered, the cancer prevention or treatment effect is superior to when PROTAC is administered alone.

The term “subject” in the present disclosure refers to all animals, including humans, that are likely to develop the cancer described above or have been affected thereby. By administering the composition of the present disclosure to a subject, cancer may be ameliorated or treated.

The method may be administered in combination with a known composition or other pharmaceutical composition that has a cancer prevention or treatment effect, may be administered simultaneously, separately, or sequentially, and may be administered in single or multiple doses. Considering all of these factors, it is important to administer an amount that can achieve the maximum effect with the minimum amount without side effects, and this could be easily determined by a person skilled in the art.

Another aspect of the present disclosure provides use of USP14 inhibitors and PROTAC for the production of drugs for preventing or treating cancer.

The “cancer,” “prevention,” “treatment,” “USP1 4 inhibitor,” “PROTAC,” etc. may be within the described scope.

Hereinafter, preferred embodiments are presented to help understanding of the present disclosure. However, the following examples are provided only to make the present disclosure easier to understand, and the content of the present disclosure is not limited by the following examples.

### EXAMPLE

### Example 1. Experiment Preparation

### 1-1. Reagents and Antibodies

The reagents and antibodies used in the present disclosure were commercially available and readily available. Specifically, the compounds used in the present disclosure were those shown in Table 1 below, and the antibodies shown in Table 2 below were used.

TABLE 1

| | |
|---|---|
| Compound | Sources |

USP14 inhibitor

| | |
|---|---|
| IU1, IU1-47 | Enamine (custom |
| | synthesized) |

PROTAC

| | |
|---|---|
| CDK9 PROTAC | Tocris |
| BRD4 PROTAC &amp; | Tocris |
| inactive form | |
| AR PROTAC | MedChemExpress |
| BTK PROTAC | MedChemExpress |
| SHP-2 PROTAC | Glixx Laboratory Inc |

USP7 inhibitor

| | |
|---|---|
| USP7/USP47 dual inhibitor | MedChemExpress |
| USP7 specific inhibitor | MedChemExpress |

Pan DUB inhibitor

| | |
|---|---|
| PR-619 | Enamine |

Proteasome inhibitor

| | |
|---|---|
| MG132 | Selleckchem |

Autophagy inhibitor

| | |
|---|---|
| Bafilomycin A1 | SantaCruz Chem |

TABLE 2

| | |
|---|---|
| Antibodies | Sources |

Primary antibodies

| | |
|---|---|
| CDK9 Rabbit mAb | Cell Signaling Technologies |
| BRD4 Rabbit mAb | (CST) |
| AR Rabbit mAb | |
| BTK Rabbit mAb | |
| SHP-2 Rabbit mAB | |
| PARP | |
| Caspase-3 Full Length | |
| Caspase-3 Cleaved | |
| γH2A.X | |
| CRBN | |
| MCL-1 | Santa Cruz Biotechnology |
| CRBN C-term | ProSci |
| USP14 | Abcam |

1-2. Compound dissolution, storage and dispensing The compounds that were synthesized in powder form and used for the experiments of the present disclosure, were dissolved at a concentration of 50 mM to 100 mM using anhydrous DMSO (Sigma) as a solvent. The dissolved compounds were used immediately in the experiment through serial dilution, or stored in small aliquots at −80° C. before use in this experiment to minimize the freeze-thaw process. When multi-dispensing of a very small amount of a compound was required, precision dispensing was performed using a custom-made high-density repetitive screening automated workstation (customized Biomek i5 HDR system, Beckman Coulter) and a pin tool.

### 1-3. Cell Culture

HeLa cells (cervical cancer cells) and LNCaP cells (prostate cancer cells) that were used for the experiments of the present disclosure were obtained from the American Type Culture Collection (ATCC), and were cultured in Dulbecco's Modified Eagle's Medium (DMEM, 1×) or RPMI-1640 (1×) medium, each containing 10% FBS, 1% penicillin/streptomycin and 1% L-glutamine, in a 37° C., 5% CO₂ cell incubator. For experiments with PROTAC and deubiquitinase (DUB) inhibitors such as IU1 and IU1-47, 5×10⁵ (HeLa) or 1×10⁶ (LNCaP) cells were seeded into each well of a 12-well plate (Corning, #3513) one day before inhibitor treatment and then cultured in an incubator. Twenty-four hours after incubation, each well was replaced with medium containing serial dilutions of the compound and incubated under the same conditions for no more than 24 hours. As for all controls, the same amount of DMSO, which is a vehicle, was used for that of the compound treated with the experimental groups.

### 1-4. Western Blotting

To prepare samples for Western blotting, HeLa and LNCaP cells were washed once with ice-cold PBS (1×) and lysed using RIPA buffer (ROCKLAND, MB-030-050) supplemented with protease inhibitor cocktail (Roche), 1 mM PMSF, and 1 mM DTT, or 1% SDS lysis buffer [1% (w/v) SDS, 10 mM Tris-HCl, 1 mM EDTA, pH 8.0]. All lysates were quantified by BCA protein assay (Thermo Fisher Scientific), and then 15 to 30 μg of each lysate was run by SDS-PAGE. Afterwards, the experiment was performed in the following order: transfer to PVDF membrane, treatment with a blocking solution (5% BSA or 5% non-fat dry milk in 1× TBST), treatment with designated primary antibody, and treatment with HRP-linked secondary antibody. Between treatments, washing was performed several times with excess 1× TBST. Each membrane was image-analyzed and quantified for protein signals using ChemiDoc (iBright750, Invitrogen).

### 1-5. Cell Viability Measurements

To measure cell viability by treatment with the compound of the present disclosure, HeLa and LNCaP cells were seeded in a 96-well plate (SPL, #33396) at 500 cells/well (HeLa) or 3000 cells/well (LNCaP). The cells were seeded and cultured in an incubator at 37° C. and 5% CO₂. Twenty four hours later, the medium was replaced with compound-containing medium and incubated under the same conditions in an incubator for 48 hours. Afterwards, cell viability was measured through luminescence analysis in an Envision microplate reader (Perkin Elmer) using the ATP-lite 1 step assay kit (Perkin Elmer).

### 1-6. Measurement of Cancer Cell Migration

To measure cell migration of the cancer cell population by treatment with the compound of the present disclosure, HeLa cells were seeded at 5×10⁴ cells/well in a 35 mm μ-dish (Ibidi, #81156) equipped with a silicone chamber, and the cells were cultured in an incubator at 37° C. and 5% CO₂ until the cells grew up to the density of 100% in the chamber. Afterwards, the silicone chamber was removed using a sterilized forcep, washed with 1 ml of 1×PBS (Corning), and replaced with 2 ml of compound-containing medium, and the cells were further cultured in an incubator at 37° C. and 5% CO₂. Microscopic images were taken using an inverted fluorescence microscope (Olympus) through a 10× magnification lens every 12 hours, and the width of the intercellular region was measured using ImageJ software.

### 1-7. Measurement of Cancer Cell Colony Formation

To measure cancer cell colony formation by treatment with the compound of the present disclosure, HeLa cells were seeded at 1000 cells/well in a 96-well plate (SPL, #33396) and cultured in an incubator at 37° C. and 5% CO₂ for two days. Afterwards, the medium was replaced with a compound-containing medium, and then the cells were further cultured in the incubator under the same conditions for one week. The cultured cancer cells at the end were stained with 0.25% crystal violet staining solution containing 20% methanol for 1 hour, and then washed with ultrapure distilled water and dried at room temperature for one day. Cell images were captured using a camera or iBright CL750 (Thermo Fisher), the number of colonies was analyzed using iBright analysis software, and the size of colonies was analyzed using ImageJ software.

### 1-8. Data Quantification and Analysis

Data obtained through Western blotting were quantified using iBright analysis software, and the levels of all target proteins were normalized with GAPDH. After quantification and normalization, DC₅₀ values of the compounds used in the experiments of the present disclosure were derived through nonlinear regression using Graph Pad Prism software.

The results measured in the experiments to confirm cell viability were initially processed using Excel, and then the IC₅₀ values were derived through nonlinear regression analysis using Prism software.

### Example 2. Confirmation of the Effect of IU1 on Enhancing the Protein Degradation Capability of PROTAC

### 2-1. Confirmation of Protein Degradation Capability of PROTAC

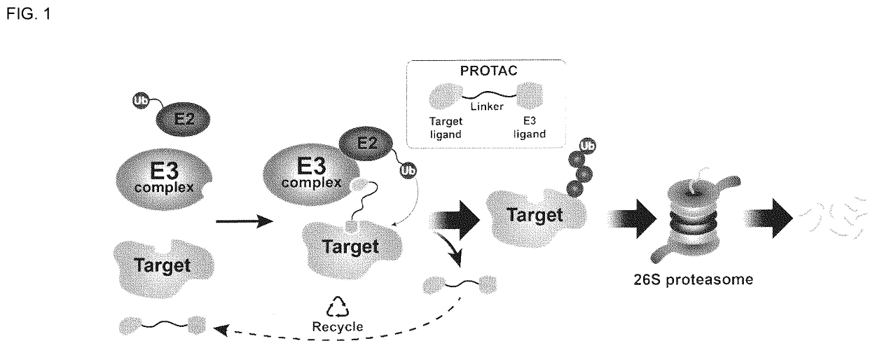

PROTAC is a targeted protein degradation (TDP) technique that uses the ubiquitin-proteasome system (UPS), which is an inherent protein degradation mechanism within cells. The specific mechanism is shown in FIG. 1.

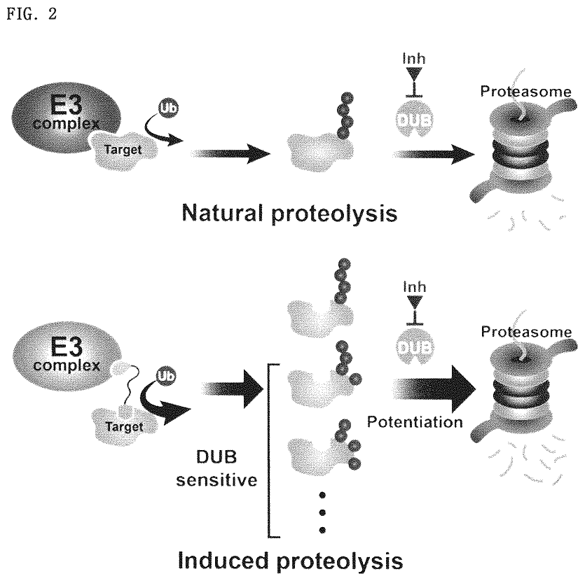

The inventors of the present specification anticipated as follows: ubiquitination of substrates by PROTAC would result in the formation of various types and shapes of poly-Ub chains or multi-Ub chains, many of which would be deubiquitinated by deubiquitinases, including USP14, and therefore inhibition of deubiquitinases would enhance the protein degradation effect of PROTAC, as shown in FIG. 2. Based on this anticipation, the inventors performed the detailed experiments as following.

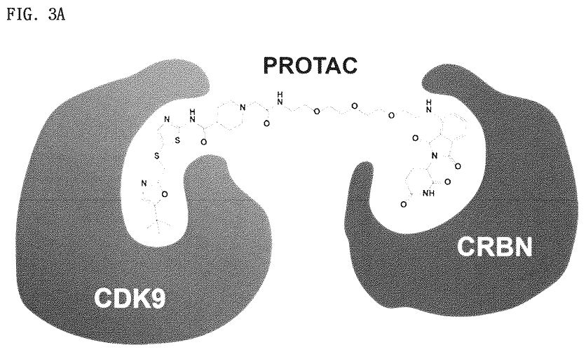

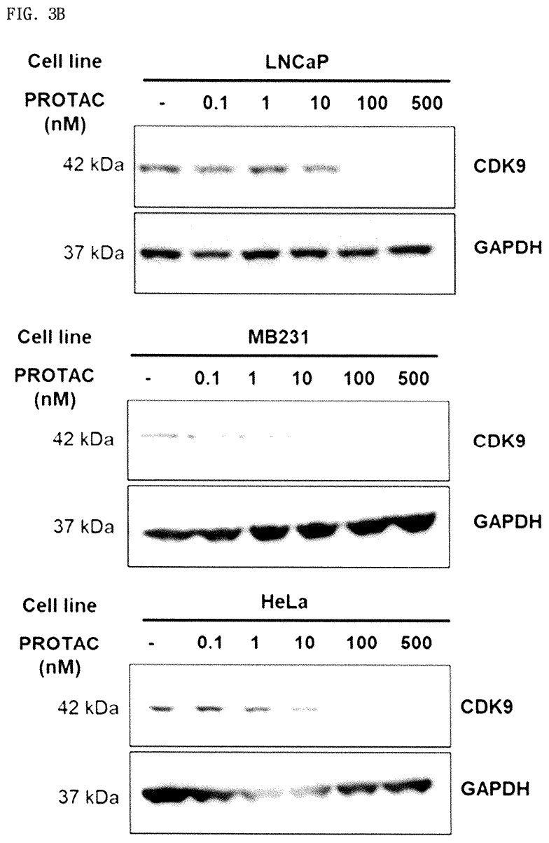

First, in order to confirm the actual protein degradation effect of PROTAC, which has been reported to act in the form shown in FIG. 3A to degrade CDK9 protein (hereinafter referred to as CDK9 PROTAC), the protein degradation capability test was performed using breast cancer (MB231), prostate cancer (LNCaP), and cervical cancer (HeLa) cell line.

As a result, as shown in FIG. 3B, it was confirmed that CDK9 PROTAC, which is known to degrade CDK9 protein, showed CDK9 protein degradation capability in all three types of cancer cell lines, consistent with previously reported results.

### 2-2. Confirmation of the Effect of IU1 on Enhancing the Protein Degradation Capability of PROTAC

The effect of IU1, an inhibitor of deubiquitinase USP14, on enhancing the protein degradation capability of CDK9 PROTAC was confirmed under the same conditions as in Example 2-1 above.

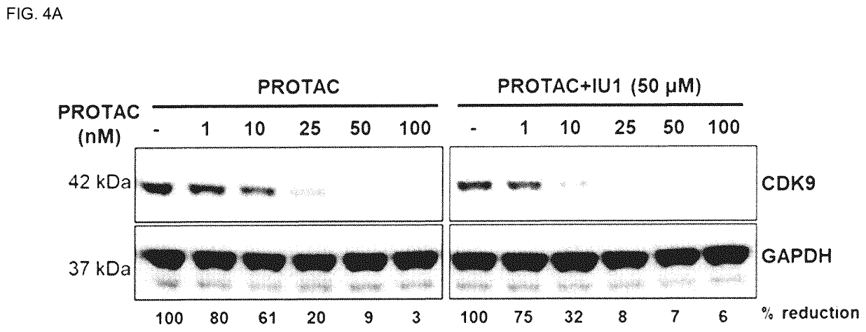

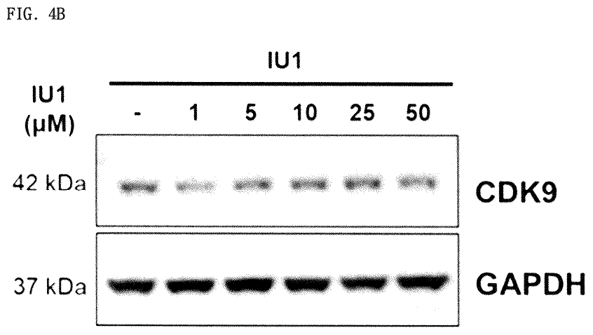

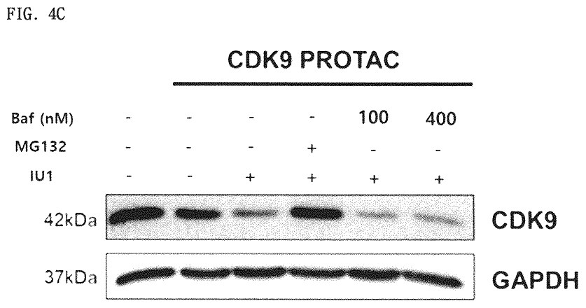

Specifically, as a result of treating HeLa cells with IU1 and CDK9 PROTAC, as shown in FIG. 4A, the CDK9 protein degradation effect was substantially increased depending on the treatment concentration (PROTAC: 1 nM, 10 nM, 25 nM, 50 nM, and 100 nM, IU1: 50 μM). As shown in FIG. 4B, it was confirmed that when treated with IU1 alone (maximum IU1 treatment concentration: 50 μM), degradation of CDK9 protein did not occur.

In addition, as shown in FIG. 4C, CDK9 PROTAC was treated with or without treatment with IU1, MG132, which is a proteasome inhibitor, or bafilomycin A1 (Baf), which is an autophagy inhibitor.

As a result, it was confirmed that when MG132, a proteasome inhibitor, was treated together with PROTAC and IU1, the level of CDK9 protein was recovered similar to the control group, whereas when treated with bafilomycin A1, the degradation of CDK9 protein by the treatment with PROTAC and IU1 was still observed.

Accordingly, the inventors of the present specification found that when PROTAC and IU1 were treated simultaneously, degradation of the target protein occurred in a proteasome-dependent manner, but not a lysosome-dependent manner.

### 2-3. Confirmation of Concentration-Dependent Protein Degradation Effect of IU1 and PROTAC

The enhancement of the protein degradation effect of CDK9 PROTAC by a deubiquitinase inhibitor IU1 depending on the concentrations, was specifically confirmed.

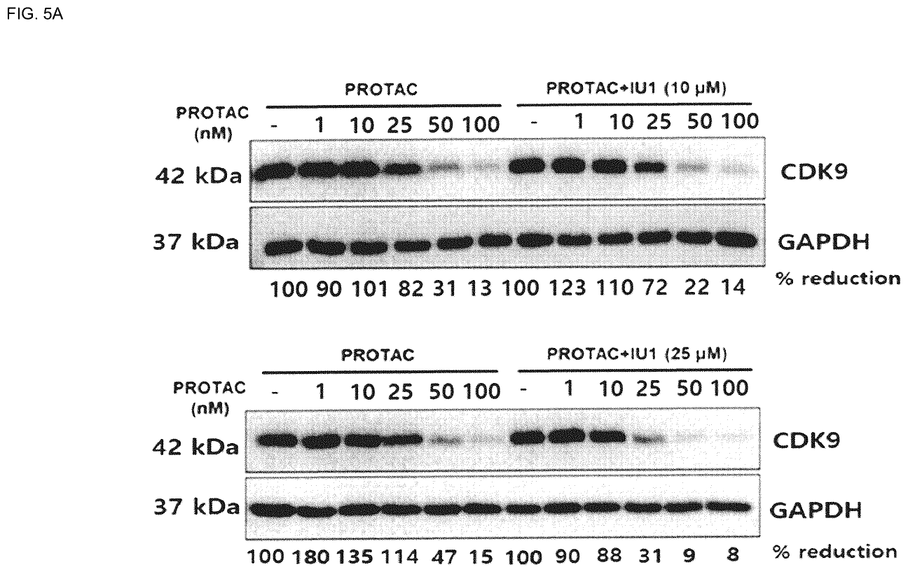

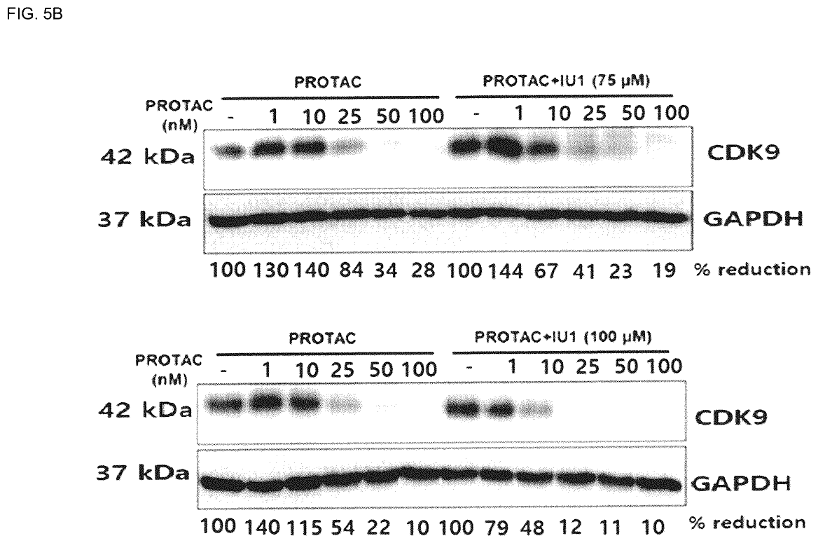

First, each concentration of IU1 (10 μM, 25 μM, 75 μM, and 100 μM) was fixed, and then the protein degradation effect of PROTAC was identified under concentration-dependent conditions (1 nM, 10 nM, 25 nM, 50 nM, and 100 nM) of CDK9 PROTAC. As shown in FIGS. 5A and 5B, it was confirmed that IU1 enhanced the CDK9 degradation effect by CDK9 PROTAC in a PROTAC concentration-dependent manner at all concentrations except for the IU1 concentration at 10 μM.

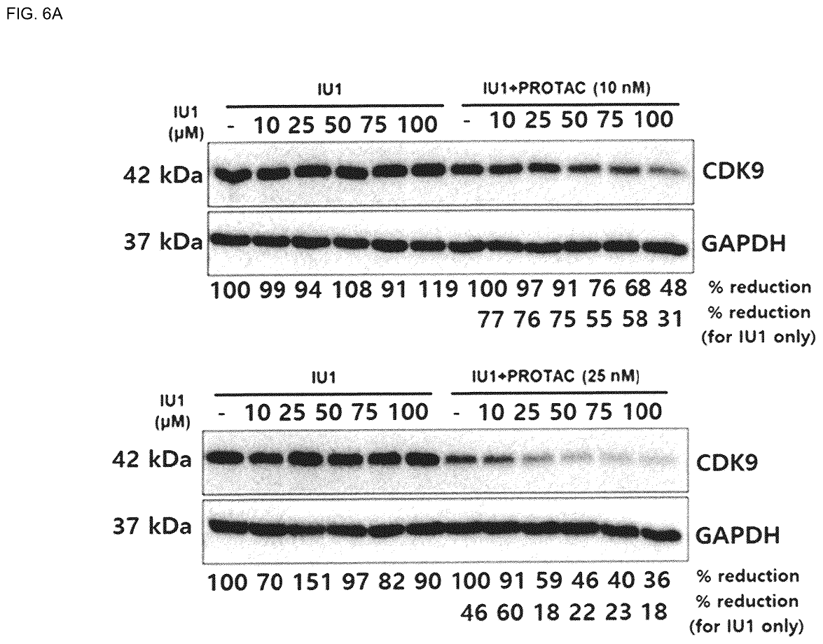

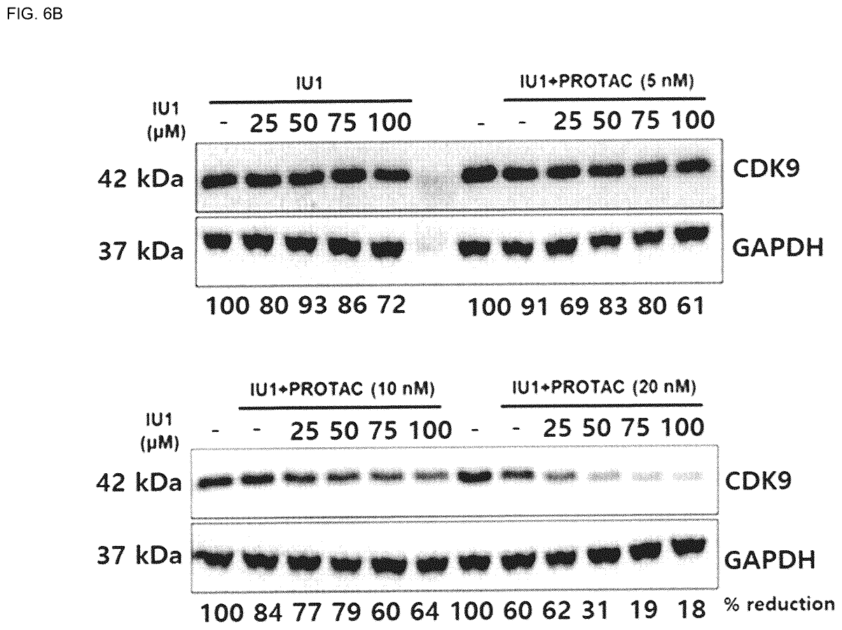

In addition, each concentration of CDK9 PROTAC (5 nM, 10 nM, 20 nM, and 25 nM) was fixed, and then the protein degradation effect of PROTAC was identified under IU1 concentration-dependent conditions (10 μM, 25 μM, 50 μM, 75 μM, and 100 μM). As shown in FIGS. 6A and 6B, it was confirmed that the protein degradation effect was enhanced in an IU1 concentration-dependent manner at all concentrations.

2-4. Measurement of DC₅₀ Value of Protein by Treatment of IU1 and PROTAC in Combination

In HeLa cells, the concentration of PROTAC was increased in the control group treated with vehicle (DMSO) and the group treated with IU1 (0.1 nM, 1, 5 nM, 10 nM, 20 nM, 30 nM, 50 nM, 75 nM, 100 nM, and 250 nM) and Western blotting was performed to confirm the level of CDK9 protein.

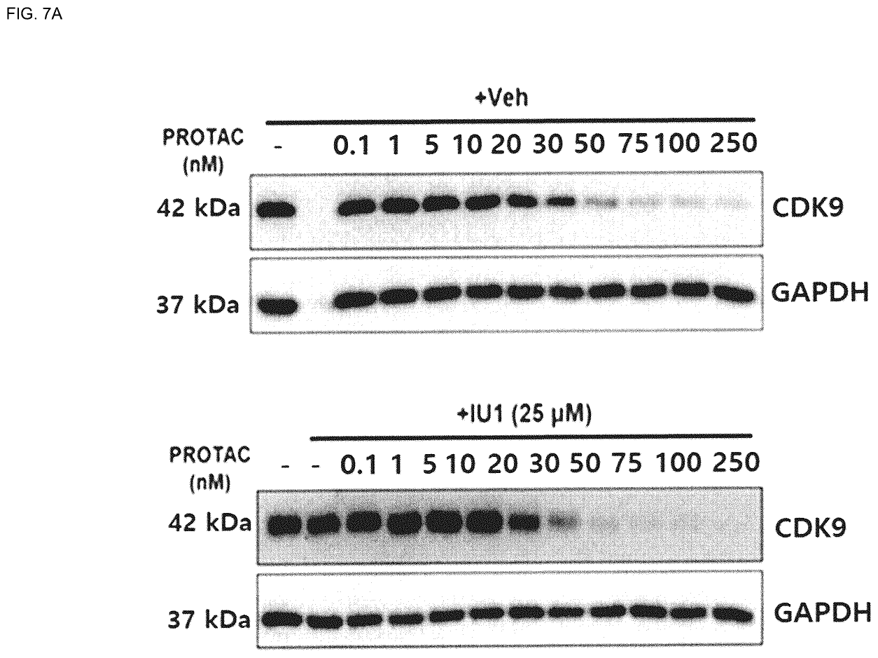

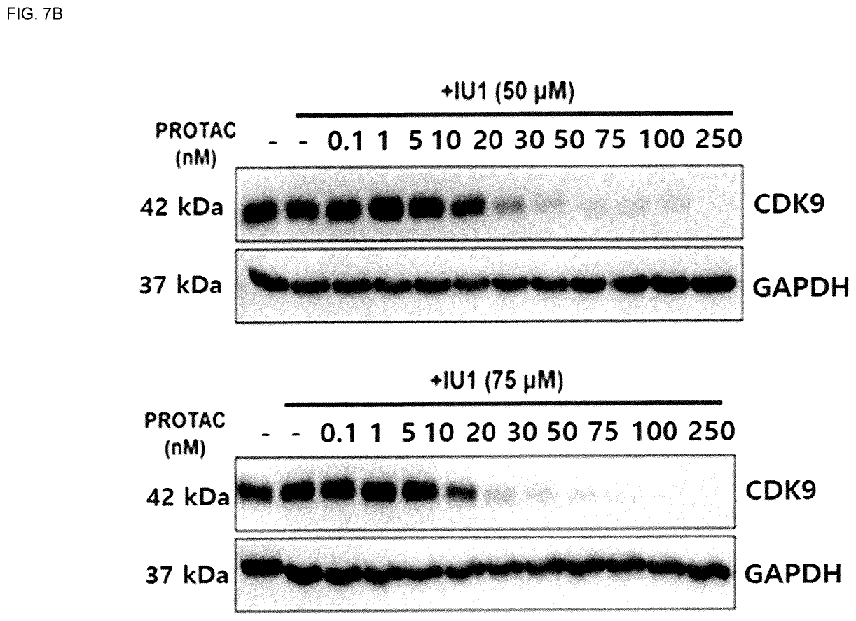

As a result, as shown in FIGS. 7A and 7B, it was confirmed that the group treated with IU1, especially the group treated with a high concentration of IU1, highly enhanced the CDK9 protein degradation capability by CDK9 PROTAC compared to the control group.

In addition, these results were quantified and each DC₅ value of CDK9 PROTAC (the concentration of PROTAC drug at which 50% of target protein degradation occurs) was measured.

As a result, as shown in FIG. 7C, it was confirmed that the DC₅₀ value of CDK9 PROTAC was significantly decreased as the IU1 concentration was increased. Accordingly, it was confirmed that when IU1 was treated in combination with CDK9 PROTAC, the CDK9 protein degradation capability of CDK9 PROTAC was significantly increased.

For example, it was confirmed through the DC₅₀ value that when treated with 75 μM of IU1 in combination, the protein degradation capability of CDK9 PROTAC was enhanced by about 5 times compared to the control group treated with vehicle.

### Example 3. Confirmation of Cancer Cell Viability by Treatment with IU1 and PROTAC in Combination

In order to confirm whether the anticancer effect of PROTAC on cancer cells is substantially improved through the enhanced degradation effect of CDK9 protein by treatment with IU1 and CDK9 PROTAC in combination, which was confirmed in Example 2, HeLa cells were treated with IU1 and CKD9 PROTAC, and then the growth rate of cancer cells was confirmed.

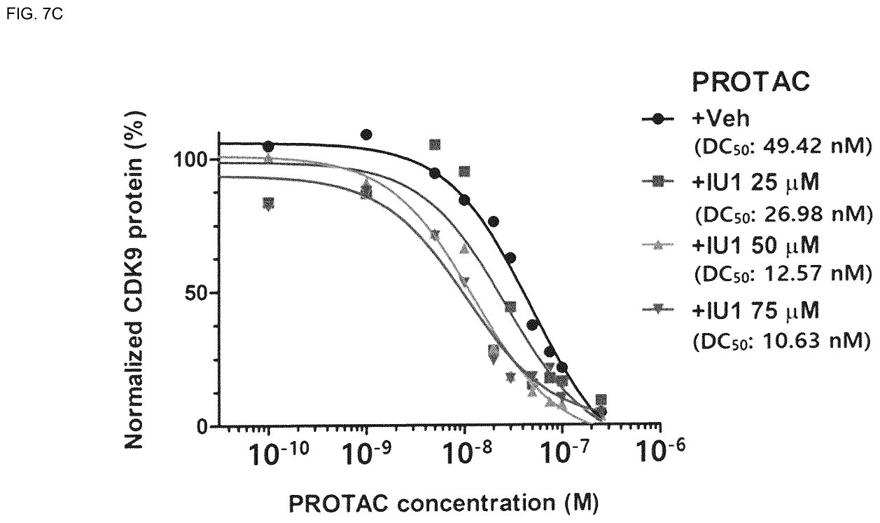

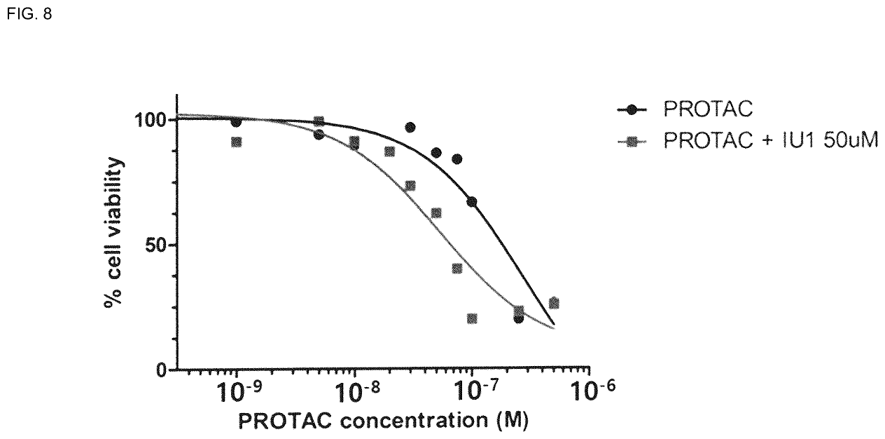

As a result, as shown in FIG. 8, it was confirmed that when IC₅₀ (the concentration of PROTAC drug at which 50% of cell growth inhibition occurs) was measured, the anticancer effect of CDK9 PROTAC was significantly increased by IU1 treatment in combination.

### Example 4. Confirmation of Protein Degradation Capability of PROTAC in LNCaP Cells Enhanced by IU1

In order to confirm that the enhancement of the PROTAC protein degradation effect by IU1 according to the present disclosure is effective not only in the HeLa cell line, which is a cervical cancer cell line, but also in other carcinomas, the LNCaP cell line, which is a prostate cancer cell line, was treated with IU1 and CDK9 PROTAC in combination and the protein degradation effect was confirmed.

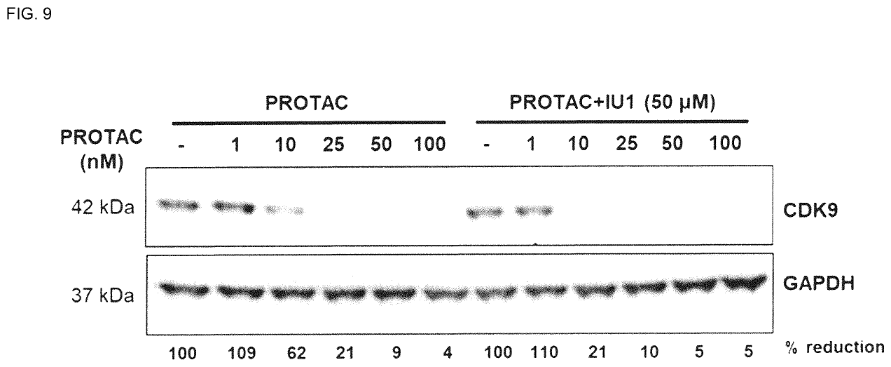

As a result, as shown in FIG. 9, it was confirmed that the degradation effect for CDK9 protein was significantly increased when IU1 and CDK9 PROTAC were treated in combination compared to when CDK9 PROTAC was treated alone.

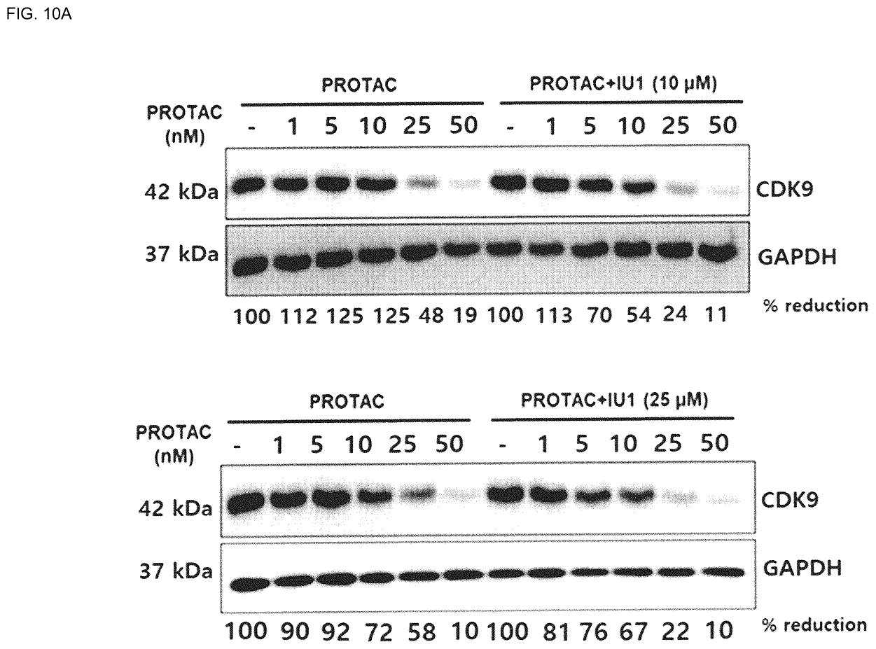

In addition, regarding this effect, as performed in Example 2-3, each concentration of IU1 was fixed (10 μM, 25 μM, 75 μM, and 100 μM), and then the protein degradation effect of PROTAC was observed under concentration-dependent conditions (1 nM, 5 nM, 10 nM, 25 nM, and 50 nM) of CDK9 PROTAC. As a result, it was confirmed that as shown in FIG. 10A and FIG. 10B, at all concentrations, IU1 enhanced the CDK9 degradation effect by CDK9 PROTAC in a PROTAC concentration-dependent manner.

Through these results, the inventors of the present specification confirmed that the effect of the USP14 inhibitor of the present disclosure to enhance the protein degradation capability of PROTAC is not limited to specific cancer types, but can be applied to all general cancer types.

### Example 5. Confirmation of Effect of Enhancing Protein Degradation Capability of PROTACs other than CDK9 PROTAC by IU1

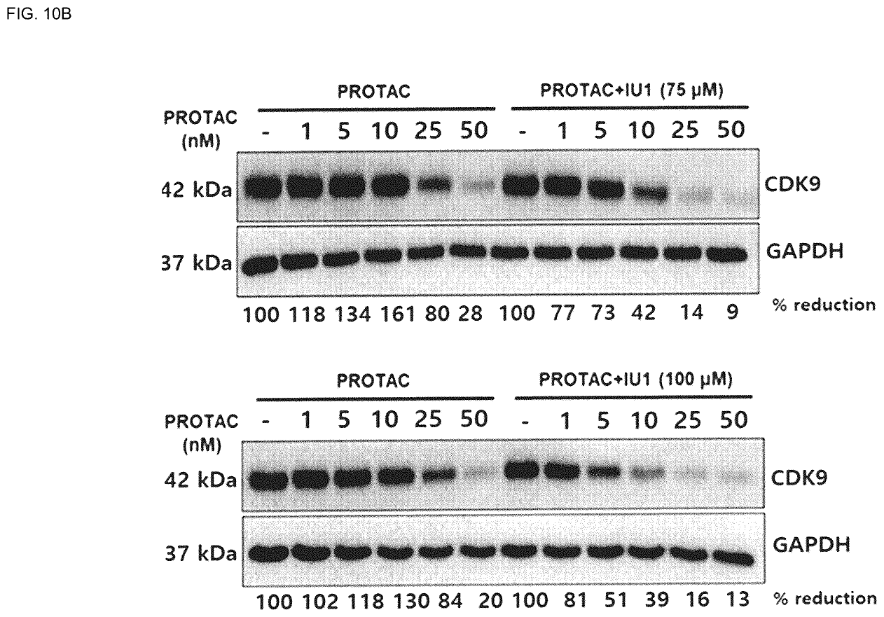

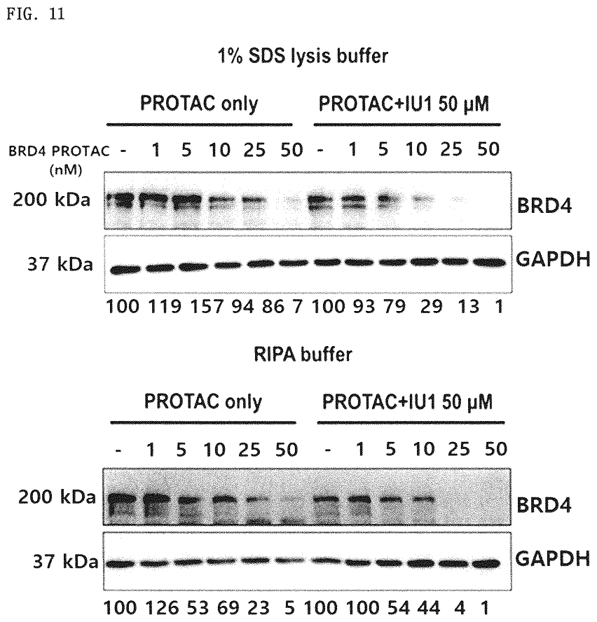

In order to determine whether the USP14 inhibitor of the present disclosure can enhance protein degradation capability for PROTACs other than CDK9 PROTACs, which were confirmed through experiments in Examples 2 and 4, it was confirmed whether the effect of degradation of BRD4 protein by BRD4 PROTAC was enhanced by IU1 treatment. To this end, the control group treated with BRD4 PROTAC alone was compared with the experimental group treated with IU1 and BRD4 PROTAC in combination by analyzing in 1% SDS lysis buffer or RIPA buffer. As a result, the treatment with 50 μM of IU1 in combination with BRD4 PROTAC in both buffers resulted in a concentration-dependent enhancement of the BRD4 protein degradation effect of PROTAC, as shown in FIG. 11.

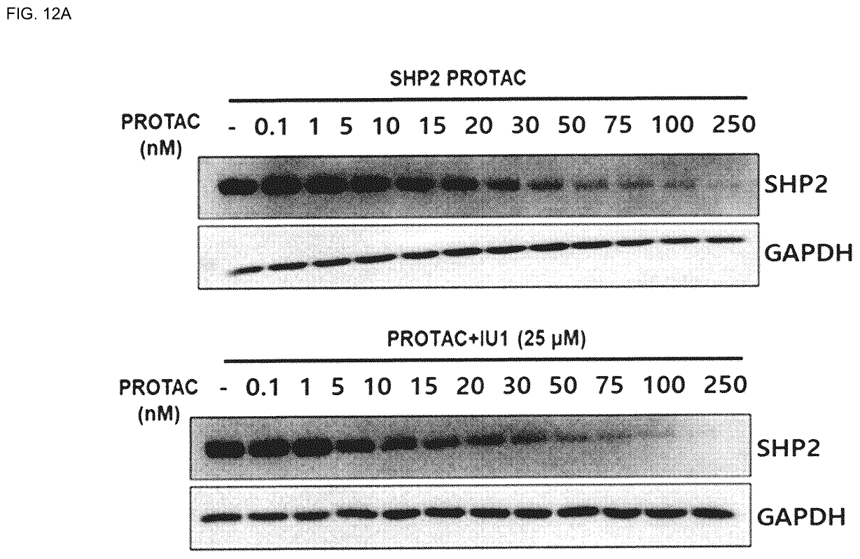

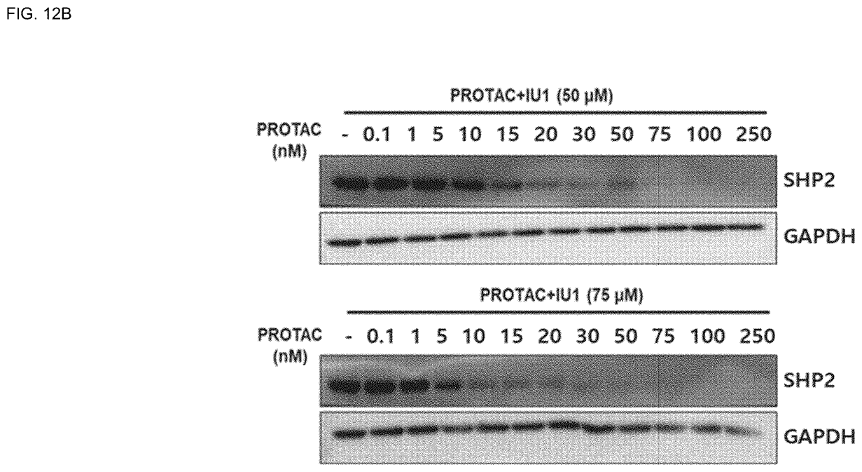

In addition, for SHP-2 PROTAC, which is another VHL-based PROTAC other than BRD4 PROTAC, it was confirmed whether the degradation effect for SHP-2 protein was enhanced under experimental conditions similar to those in the experiment in FIG. 7. As a result, as shown in FIGS. 12A and 12B, the treatment with IU1 in combination with SHP-2 PROTAC resulted in a SHP-2 PROTAC concentration-dependent enhancement of SHP-2 protein degradation.

Furthermore, based on the above results, the inventors of the present specification have found that the effect of the USP14 inhibitor of the present disclosure on enhancing the protein degradation of PROTAC is not limited to CRBN-based E3 ligase PROTACs, such as CDK9 PROTAC, but is also applicable to VHL-based E3 ligase PROTAC.

Example 6. Confirmation of the Effect of USP14 Inhibitor other than IU1 on Enhancing the Protein Degradation Capability of PROTAC

Since the previous studies have shown that in addition to IU1, the USP14 deubiquitinase inhibitor identified in Examples 2 to 4 of the present disclosure, other IU1 series inhibitors, including IU1-47, inhibited USP14, it was also confirmed whether, in addition to IU1, IU1-47, which is another IU1 series USP14 inhibitor, could also enhance the protein degradation capability of PROTAC in the same manner as IU1. For this purpose, the level of CDK9 protein degradation was identified under conditions where each concentration (10 μM, 20 82 M, and 30 μM) of IU1-47 and vehicle (DMSO) group were fixed and CDK9 PROTAC was treated at different concentrations (0.1 nM, 1 nM, 5 nM, 10 nM, 20 nM, 30 nM, 50 nM, 75 nM, 100 nM, and 250 nM).

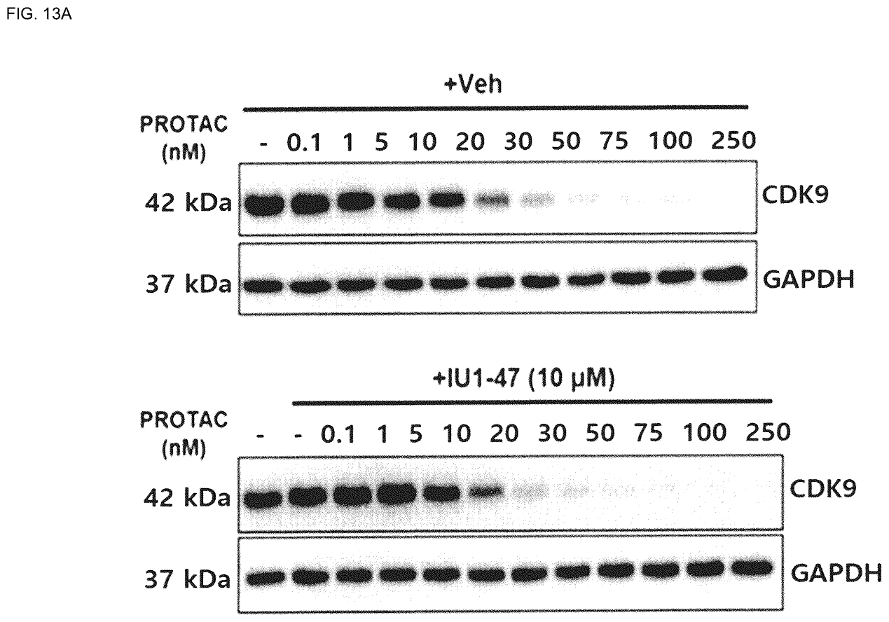

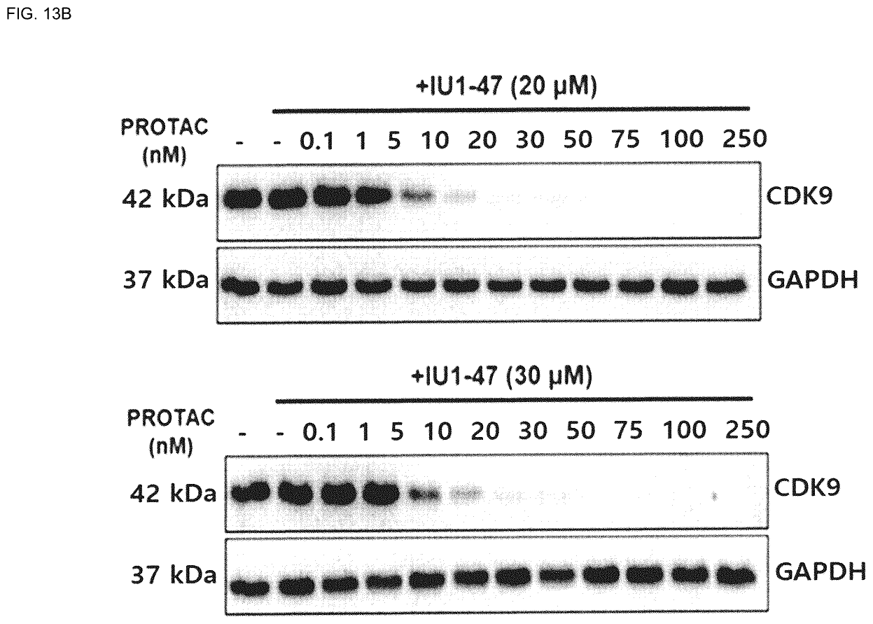

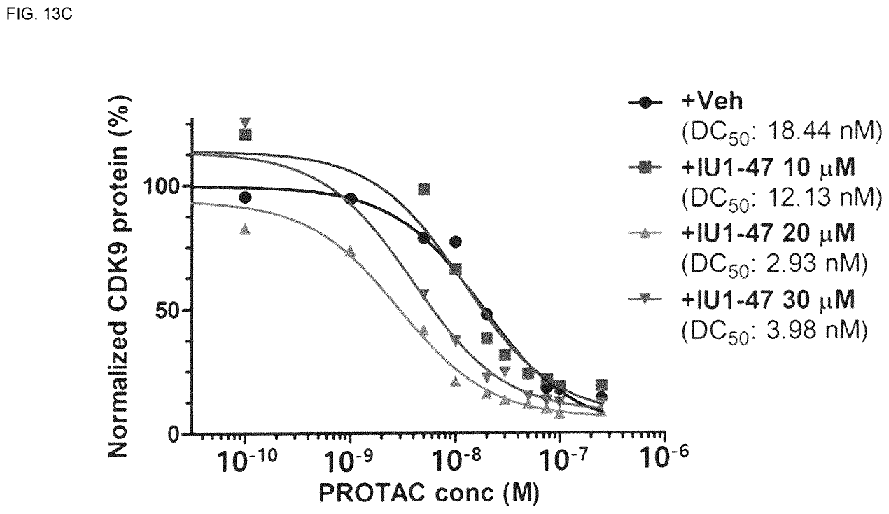

As a result, as shown in FIGS. 13A and 13B, it was confirmed that compared to the control group treated with vehicle alone, the protein degradation capability of PROTAC was significantly enhanced at all concentrations of IU1-47.

Furthermore, when the data obtained in FIGS. 13A and 13B above were quantified in FIG. 13C, and DC₅₀ values were specifically determined, it was found that the treatment with 20 μM IU1-47, which is an IU1 series deubiquitinase inhibitor, in combination with CDK9 PROTAC, enhanced the protein degradation of PROTAC by about 6 times compared to the treatment with CDK9 PROTAC alone.

Furthermore, to determine whether the protein degradation enhancing effect of IU1-47 on PROTAC as described above is concentration dependent in a similar manner to that of IU1, each concentration of PROTAC was fixed (5 nM, 10 nM, and 20 nM), and the level of protein degradation upon treatment with IU1-47 in combination with PROTAC was identified at varying concentrations of IU1-47.

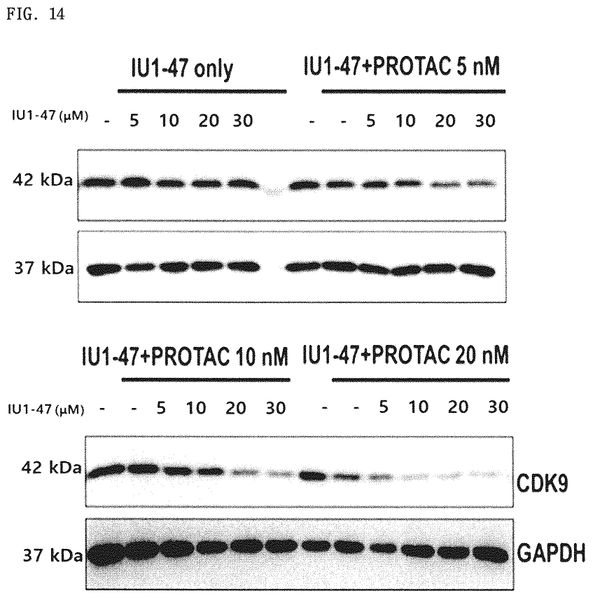

As shown in FIG. 14, the treatment with IU1-47 alone did not result in any degradation of CDK9, but the co-treatment thereof with CDK9 PROTAC significantly enhanced the degradation of PROTAC in a concentration-dependent manner.

### Example 7. Confirmation of Enhanced Protein Degradation Capability of PROTAC Over Time in Cells by IU1

It was confirmed that in HeLa cells treated with CDK9 PROTAC (100 nM) alone or in combination with IU1 (50 μM), the effect of the IU1 compound on enhancing protein degradation capability of CDK9 PROTAC was time-dependent (0 h, 1 h, 3 h, 6 h, 12 h, and 24 h).

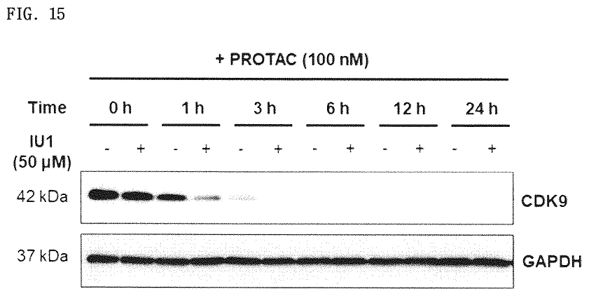

The results showed that as shown in FIG. 15, the treatment with IU1 in combination accelerated the degradation effect of PROTAC.

### Example 8. Determination of the Effect of Deubiquitinase Inhibitors other than the IU1 Series on Enhancing the Protein Degradation Capability of PROTAC

The effect of PR-619 compound, which is a non-specific deubiquitinase inhibitor (pan DUB inhibitor), on enhancing the protein degradation capability of CDK9 PROTAC was compared with the effect of IU1-47 after setting PR-619 compound at different concentration conditions in HeLa cells.

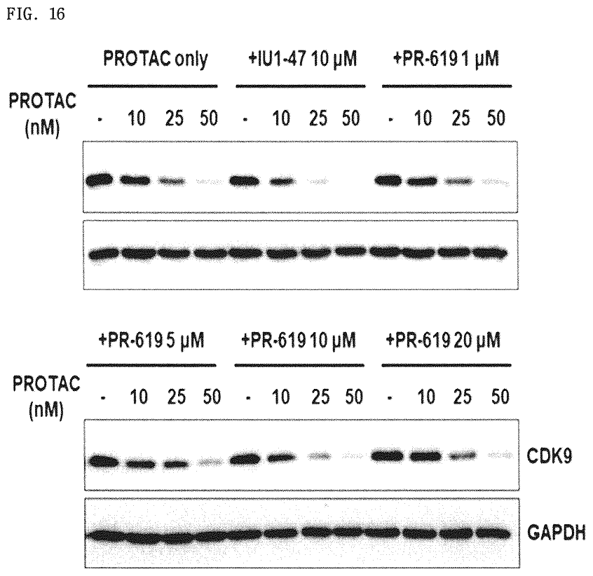

As a result, as shown in FIG. 16, IU1-47 significantly enhanced the degradation effect of PROTAC, while PR-619 did not show the effect of enhancing the protein degradation of PROTAC at all treatment concentrations.

In addition, in HeLa cells, USP7/47 dual inhibitor (hereinafter referred to as P50429), which is a dual deubiquitinase inhibitor of USP7 and USP47, was set to have the concentrations of 1 μM and 10 μM, and treated in combination with CDK9 PROTAC to identify the effect of enhancing the protein degradation capability of PROTAC. The enhancement effect of P50429 was compared with that of IU1-47.

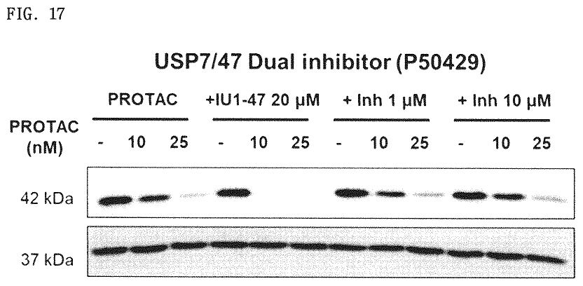

As a result, as shown in FIG. 17, IU1-47 (20 μM) significantly enhanced the degradation effect of PROTAC, while P50429 did not enhance the degradation effect of PROTAC.

Accordingly, the inventors of the present specification found that the effect of enhancing the degradation of PROTAC by deubiquitinase inhibitors was achieved not through non-specific deubiquitinase inhibitors or USP7/47 deubiquitinase inhibitors, but through a specific mechanism by IU1 series inhibitors.

### Example 9. Confirmation of Anticancer Effect by Combined Treatment of IU1-Series Deubiquitinase Inhibitor and PROTAC

Whether the PROTAC anticancer effect had been substantially improved by the degradation of the CDK9 protein enhanced by the combined treatment of the IU1-series compound and CDK9 PROTAC, which was confirmed in Examples as described above, was confirmed with respect to cancer cell migration, which is closely correlated with cancer cell metastasis. To this end, HeLa cells were treated with CDK9 PROTAC (40 nM) and IU1-47 (20 μM) compounds separately or in combination, and the migration of HeLa cells was confirmed every 12 hours.

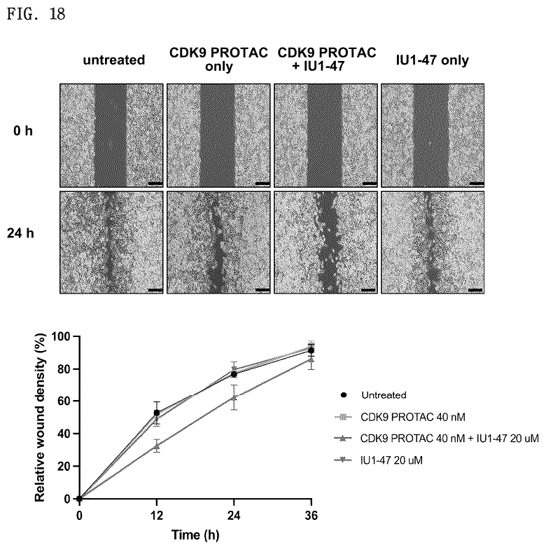

As a result, as shown in FIG. 18, compared to the condition in which CDK9 PROTAC or IU1-47 alone was treated, when IU1-47 and CDK9 PROTAC were used in combination for treatment, the migration or wound healing effect of HeLa cells was significantly inhibited.

As another example to confirm the anticancer effect by the degradation of CDK9 protein enhanced by the combined treatment of the IU1-series compound and CDK9 PROTAC, which was confirmed in Examples as described above, different concentrations of CDK9 PROTAC (10, 25 , 50, 75, 100 nM) and IU1-47 (20 μM) compounds were used separately or in combination for treatment, and one week later, the degree of colony formation of HeLa cancer cells by the effect of enhancing CDK9 PROTAC protein degradation capability by IU1-series compound, was confirmed.

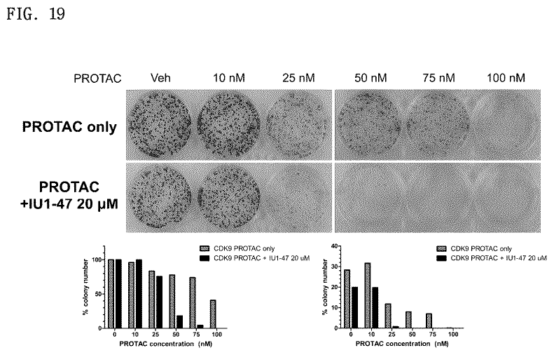

As a result, as shown in FIG. 19, compared to the condition in which CDK9 PROTAC or IU1-47 alone was treated, when IU1-47 and CDK9 PROTAC were used in combination for treatment, colony formation of HeLa cancer cells was significantly inhibited.

From the results above, the inventors have confirmed that regarding the IU1 series among the deubiquitinase USP14 inhibitors, not only IU1, but also IU1-47, have the effect of enhancing the protein degradation capability of PROTAC, thereby significantly enhancing the anticancer effect of PROTAC when the compounds of the present disclosure are treated in combination with PROTAC.

The description of the present disclosure stated above is for illustrative purposes, and a person skilled in the art to which the present disclosure pertains could understand that it could be easily modified into other specific forms without changing the technical concept or essential features of the present disclosure. Therefore, the embodiments described above should be understood in all respects as illustrative and not restrictive.

1. A pharmaceutical composition for preventing or treating cancer, comprising a ubiquitin-specific protease 14 (USP14) inhibitor and a proteolysis-targeting chimera (PROTAC).

2. The composition of claim 1, wherein the USP14 inhibitor is an IU1-series compound.

3. The composition of claim 2, wherein the IU1-series compound is selected from the group consisting of: 1[1-(4-fluorophenyl)-2,5-dimethylpyrrol-3-yl]-2-pyrrolidin-1-ylethanone; 1-[1-(4-chlorophenyl)-2,5-dimethyl-1H- pyrrol-3-yl]-2-(1-piperidinyl)ethanone; 1-(1-(4-chlorophenyl)-2,5-dimethyl-1H-pyrrol-3-yl)-2-(pyrrolidin-1-yl)ethan-1-one; 4-(3-(2-(4-hydroxypiperidin-1-yl)acetyl)-2,5-dimethyl-1H-pyrrol-1-yl)benzonitrile; 1-(1-(4-chlorophenyl)-5-methyl-1H-pyrazol-4-yl)-2-(piperidin-1-yl)ethan-1-one; 4-(2-methyl-3-(2-(piperidin-1-yl)acetyl)-1H-pyrrolo[3,2-b]pyridin-1-yl)benzonitrile; 1-(4-cyanophenyl)-2-methyl-3-(2-(piperidin-1-yl)acetyI)-1H-pyrrolo[3,2-b]pyridine-5-carbonitrile; 4-(3-(2-((2R)-2-hydroxy-7-azabicyclo[2.2.1]heptan-7-yl)acetyl)-2-methyl-5-(3-(tetrahydro-2H-pyran-4-yl)propyl)-1H-pyrrol-1- yl)benzonitrile; (E)-2-(7-azabicyclo[2.2.1]heptan-7-yl)-1-(1-(4-chlorophenyl)-2-methyl-5-(4-(methylsulfonyl)but-1-en-1-yl)-1H-pyrrol-3- yl)ethan-1-one; 4-(3-(2-((1r,3r,5r,7r)-2-azaadamantan-2-yl)acetyl)-2,5-dimethyl-1H-pyrrol-1-yl)benzonitrile; and 4-(5-((E)-3-(1,3-dioxoisoindolin-2-yl)prop-1-en-1-yl)-3-(2-((2R)-2-hydroxy-7-azabicyclo[2.2.1]heptan-7-yl)acetyl)-2-methyl-1H-pyrrol-1-yl)benzonitrile.

4. The composition of claim 1, wherein the cancer is brain tumor, head and neck cancer, breast cancer, lung cancer, esophageal cancer, stomach cancer, duodenal cancer, appendix cancer, colon cancer, rectal cancer, liver cancer, pancreatic cancer, gallbladder cancer, bile duct cancer, anal cancer, renal cancer, ureteral cancer, bladder cancer, prostate cancer, penile cancer, testicular cancer, uterine cancer, ovarian cancer, vulvar cancer, vaginal cancer, or skin cancer.

5. The composition of claim 1, wherein the PROTAC targets a protein selected from the group consisting of cyclin-dependent kinase 9 (CDK9)/CDK family, bromodomain-containing protein 4 (BRD4)/bromodomain and extra-terminal (BET) family, Src homology region 2 domain-containing phosphatase-2 (SHP-2), androgen receptor (AR), estrogen receptor (ER), retinoic acid receptor (RAR), estrogen-related receptor alpha (ERRα), Bruton's tyrosine kinase (BTK), anaplastic lymphoma kinase (ALK), receptor-interacting serine/threonine-protein kinase (RIPK), MET proto-oncogene/receptor tyrosine kinase (c-Met), focal adhesion kinase (FAK), interleukin-1 receptor-associated kinase 4 (IRAK4), p38 mitogen-activated protein kinases (p38 MAPK), serum and glucocorticoid-regulated kinase (SGK), TANK-binding kinase 1 (TBK1), Kirsten rat sarcoma virus protein (KRAS), B-Raf proto-oncogene serine/threonine kinase (B-Raf), β-catenin, FK506 binding protein (FKBP), indoleamine 2,3-dioxygenase (IDO), programmed death protein 1/programmed death-ligand 1 (PD-1/PD-L1), poly[ADP-ribose]polymerase 1 (PARP1), polycomb repressive complex 2 (PRC2), epidermal growth factor receptor (EGFR), NAD-dependent deacetylase sirtuin 2 (Sirt2), human epidermal growth factor receptor 2 (HER2/ERBB2), fibroblast growth factor receptor substrate 2 (FRS2), B-cell lymphoma-extra large (BCL-XL), SWI/SNF related matrix associated actin dependent regulator of chromatin subfamily A (SMARCA), human double minute 2 homolog (HDM2), histone deacetylase (HDAC) family, breakpoint cluster region protein- tyrosine-protein kinase ABL1 (BCR-ABL), modulator of VRAC current 1 (MCL1), Fms related receptor tyrosine kinase 3 (FLT-3), transcription factor STAT3 (STAT3), Myc family, and Brg/Brahma-associated factors (BAF complex).

6. The composition of claim 1, wherein the USP14 inhibitor enhances the protein degradation effect of PROTAC.

7. A pharmaceutical composition for enhancing the effect of preventing or treating cancer, used with proteolysis-targeting chimera (PROTAC) in combination, the pharmaceutical composition comprising a ubiquitin-specific protease 14 (USP14) inhibitor as an active ingredient.

8. The composition of claim 7, wherein the USP14 inhibitor is an IU1-series compound.

9. The composition of claim 7, wherein the IU1-series compound is selected from the group consisting of: 1-[1-(4-fluorophenyl)-2,5-dimethylpyrrol-3-yl]-2-pyrrolidin-1-ylethanone; 1-[1-(4-chlorophenyl)-2,5-dimethyl-1H-pyrrol-3-yl]-2-(1 -piperidinyl)ethanone; 1-(1-(4-chlorophenyl)-2,5-dimethyl-1 H-pyrrol-3-yl)-2-(pyrrolidin-1-yl)ethan-1-one; 4-(3-(2-(4-hydroxypiperidin-1-yl)acetyl)-2,5-dimethyl-1H-pyrrol-1-yl)benzonitrile; 1-(1-(4- chlorophenyl)-5-methyl-1H-pyrazol-4-yl)-2-(piperidin-1-yl)ethan-1-one; 4-(2-methyl-3-(2-(piperidin-1-yl)acetyl)-1H-pyrrolo[3,2-b]pyridin-1- yl)benzonitrile; 1 -(4-cyanophenyl)-2-methyl-3-(2-(piperidin-1-yl)acetyl)-1H-pyrrolo[3,2-b]pyridine-5-carbonitrile; 4-(3-(2-((2R)-2-hydroxy-7-azabicyclo[2.2.1]heptan-7-yl)acetyl)-2-methyl-5-(3-(tetrahydro-2H-pyran-4-yl)propyl)-1H-pyrrol-1-yl)benzonitrile; (E)-2-(7-azabicyclo[2.2.1]heptan-7-yl)-1 -(1-(4-chlorophenyl)-2-methyl-5-(4-(methylsulfonyl)but-1 -en-1-yl)-1H-pyrrol-3-yl)ethan-1-one; 4-(3-(2-((1r,3r,5r,7r)-2-azaadamantan-2-yl)acetyl)-2,5-dimethyl-1H-pyrrol-1-yl)benzonitrile; and 4-(5-((E)-3-(1,3-dioxoisoindolin-2-yl)prop-1-en-1-yl)-3-(2-((2R)-2-hydroxy-7-azabicyclo[2.2.1]heptan-7-yl)acetyl)-2-methyl-1H-pyrrol-1-yl)benzonitrile.

10. The composition of claim 7, wherein the cancer is brain tumor, head and neck cancer, breast cancer, lung cancer, esophageal cancer, stomach cancer, duodenal cancer, appendix cancer, colon cancer, rectal cancer, liver cancer, pancreatic cancer, gallbladder cancer, bile duct cancer, anal cancer, renal cancer, ureteral cancer, bladder cancer, prostate cancer, penile cancer, testicular cancer, uterine cancer, ovarian cancer, vulvar cancer, vaginal cancer, or skin cancer.

11. The composition of claim 7, wherein the PROTAC targets a protein selected from the group consisting of cyclin-dependent kinase 9 (CDK9)/CDK family, bromodomain-containing protein 4 (BRD4)/bromodomain and extra-terminal (BET) family, Src homology region 2 domain-containing phosphatase-2 (SHP-2), androgen receptor (AR), estrogen receptor (ER), retinoic acid receptor (RAR), estrogen-related receptor alpha (ERRα), Bruton's tyrosine kinase (BTK), anaplastic lymphoma kinase (ALK), receptor-interacting serine/threonine-protein kinase (RIPK), MET proto-oncogene/receptor tyrosine kinase (c-Met), focal adhesion kinase (FAK), interleukin-1 receptor-associated kinase 4 (IRAK4), p38 mitogen-activated protein kinases (p38 MAPK), serum and glucocorticoid-regulated kinase (SGK), TANK-binding kinase 1 (TBK1), Kirsten rat sarcoma virus protein (KRAS), B-Raf proto-oncogene serine/threonine kinase (B-Raf), β-catenin, FK506 binding protein (FKBP), indoleamine 2,3-dioxygenase (IDO), programmed death protein 1/programmed death-ligand 1 (PD-1/PD-L1), poly[ADP-ribose]polymerase 1 (PARP1), polycomb repressive complex 2 (PRC2), epidermal growth factor receptor (EGFR), NAD-dependent deacetylase sirtuin 2 (Sirt2), human epidermal growth factor receptor 2 (HER2/ERBB2), fibroblast growth factor receptor substrate 2 (FRS2), B-cell lymphoma-extra large (BCL-XL), SWI/SNF related matrix associated actin dependent regulator of chromatin subfamily A (SMARCA), human double minute 2 homolog (HDM2), histone deacetylase (HDAC) family, breakpoint cluster region protein- tyrosine-protein kinase ABL1 (BCR-ABL), modulator of VRAC current 1 (MCL1), Fms related receptor tyrosine kinase 3 (FLT-3), transcription factor STAT3 (STAT3), Myc family, and Brg/Brahma-associated factors (BAF complex).

12. The composition of claim 7, wherein the USP14 inhibitor enhances the protein degradation effect of PROTAC.

13. The composition of claim 7, wherein the use in combination is administering simultaneously, separately, or sequentially with PROTAC.

14. A health functional food for preventing or ameliorating cancer, comprising a ubiquitin-specific protease 14 (USP14) inhibitor and proteolysis-targeting chimera (PROTAC).

15. A method of preventing or treating cancer comprising administering, to a subject, a ubiquitin-specific protease 14 (USP14) inhibitor and proteolysis-targeting chimera (PROTAC).

16. Use of a ubiquitin-specific protease 14 (USP14) inhibitor and proteolysis-targeting chimera (PROTAC) for the production of drugs for the prevention or treatment of cancer.
